(19) United States
(12) Patent Application Publication
(10) Pub. No.: US 2021/0401898 A1
(43) Pub. Date: Dec. 30, 2021

(54) USE OF IMMORTALIZED PLACENTAL STEM CELLS IPSC/EXTRACELLULAR VESICLES TO ENHANCE THERAPEUTIC RECOVERY FROM TISSUE DAMAGE AND ISCHEMIA-REPERFUSION INJURY AND DELAYED ORGAN FUNCTION

(71) Applicant: Board of Supervisors of Louisiana State University and Agricultural and Mechanical College, Baton Rouge, LA (US)

(72) Inventors: Johnathan Steven ALEXANDER, Shreveport, LA (US); Mansoureh BARZEGAR, Lafayette, LA (US); Yuping WANG, Shreveport, LA (US); Anthony Wayne ORR, Benton, LA (US); Mabruka ALFAIDI, Shreveport, LA (US)

(73) Assignee: Board of Supervisors of Louisiana State University and Agricultural and Mechanical College, Baton Rouge, LA (US)

(21) Appl. No.: 17/228,276

(22) Filed: Apr. 12, 2021

Related U.S. Application Data
(60) Provisional application No. 62/990,332, filed on Mar. 16, 2020.

(51) Int. Cl. A61K 35/50; C12N 5/0775

(52) U.S. Cl. CPC A61K 35/50; C12N 5/0668

(57) ABSTRACT

Therapeutics and methods for treating a stroke in a human comprising administering a therapeutic to the patient, wherein the therapeutic comprises one of immortalized human placenta mesenchymal stem cells (hPMSCs) and a product from the immortalized hPMSCs. Methods and therapeutic products comprising extracellular vesicles (EVs) isolated from of immortalized hPMSCs. Therapeutics and methods of treating COVID-19 disease vascular injury comprising administering a therapeutic to the patient, wherein the therapeutic comprises one of hPMSCs and a product from the hPMSCs.

### CROSS REFERENCE TO RELATED APPLICATIONS/PRIORITY

The present invention claims priority to U.S. Provisional Patent Application No. 62/990,332 filed Mar. 16, 2020, which is incorporated by reference into the present disclosure as if fully restated herein. U.S. patent application Ser. No. 16/835,246, filed Mar. 30, 2020, and Ser. No. 17/086,315, filed Oct. 30, 2020, are incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND

87% of strokes are ‘ischemic’ in nature and are the leading cause of neurologically based morbidity in the elderly. The incidence of ischemic stroke has also been increasing among ‘younger’ (<65 years) individuals since age-related risk factors (hypertension, obesity) are increasingly found in younger patients. Additionally, ‘younger’ stroke risk factors (pregnancy, oral contraceptive use) and behavioral risks (sedentary lifestyle, alcohol, tobacco and recreational drug abuse) also contribute to an elevated stroke risk in younger populations. It was recently reported that as many as 5% of patients with COVID-19 infections developed acute ischemic stroke which appears to be a common clinical feature of COVID disease. Currently ischemic stroke treatment is limited and has risks, such as a narrow time-window (<4.5 h) for administration after onset of stroke symptoms and concern over risk for hemorrhage. This lack of highly effective and safe therapies for the critical acute phase of stroke has motivated the inventors to seek alternative therapeutic approaches which reduce cerebral injury before and beyond this timeframe.

### SUMMARY

Wherefore, it is an object of an embodiment of the present invention to overcome the above-mentioned shortcomings and drawbacks associated with the current technology.

The presently disclosed invention is related to therapeutics and methods for treating a stroke in a human comprising administering a therapeutic to the patient, wherein the therapeutic comprises one of immortalized human placenta mesenchymal stem cells (hPMSCs) and a product from the immortalized hPMSCs. According to a further embodiment, the hPMSCs are sterol medium cultured hPMSCs. According to a further embodiment, the hPMSCs were immortalized using one of a catalytic subunit of human telomerase (hTERT) and a SV40 large T antigen. According to a further embodiment, the route of administration is intraperitoneal (IP). According to a further embodiment, the therapeutic comprises hPMSCs. According to a further embodiment, the therapeutic comprises Extracellular Vesicles (EVs) isolated from the hPMSCs. According to a further embodiment, the route of administration is intravenous (IV). According to a further embodiment, the therapeutic comprises extracellular vesicles (EVs) isolated from the hPMSCs and the sterol is cholesterol. According to a further embodiment, the therapeutic contains substantially no live hPMSCs. According to a further embodiment, the therapeutic further contains further comprises one of a mas Receptor (masR) agonists and a reagent to biochemically suppress the clearance of the Ang1-7. According to a further embodiment, a dosage of the therapeutic that is administered for a human patient is one of (a) 2.0×10⁸ and 1.0×10¹⁰ EVs per 70 kg patient mass, (b) 5.0×10⁸ and 5.0×10⁹ EVs per 70 kg patient mass, (c) 1.0×10⁹ and 4.0×10⁹ EVs per 70 kg patient mass, and (d) 2.0×10⁹ EVs per 70 kg patient mass. According to a further embodiment, the stroke is one of occlusive, post-occlusive, hemorrhagic, and transient ischemic injury.

The presently disclosed invention is further related to methods and therapeutic products comprising extracellular vesicles (EVs) isolated from of immortalized human placenta mesenchymal stem cells (hPMSCs). According to a further embodiment, the hPMSCs were cultured with cholesterol for at least 12 hours before the EVs were isolated from the hPMSCs. According to a further embodiment, the EVs are packaged in units of between one of (a) 1.0×10⁸ and 5.0×10⁹ EVs, (b) 2.5×10⁸ and 2.0×10⁹ EVs, and (c) 5.0×10⁸ and 1.0×10⁹ EVs.

According to a further embodiment, the therapeutic product further comprises one of a mas Receptor (masR) agonists and a reagent to biochemically suppress the clearance of the Ang1-7.

The presently disclosed invention is further related to therapeutics and methods of treating COVID-19 disease vascular injury comprising administering a therapeutic to the patient, wherein the therapeutic comprises one of human placenta mesenchymal stem cells (hPMSCs) and a product from the hPMSCs. According to a further embodiment, the hPMSCs are immortalized. According to a further embodiment, the route of administration is intravenous (IV), the further comprises therapeutic comprises extracellular vesicles (EVs) isolated from the hPMSCs, and the therapeutic is substantially free from live hPMSCs. According to a further embodiment, the therapeutic further comprises one of a mas Receptor (masR) agonists and a reagent to biochemically suppress the clearance of the Ang1-7. According to a further embodiment, a dosage of the therapeutic that is administered for a human patient is one of (a) 2.0×10⁸ and 1.0×10¹⁰ EVs per 70 kg patient mass, (b) 5.0×10⁸ and 5.0×10⁹ EVs per 70 kg patient mass, (c) 1.0×10⁹ and 4.0×10⁹ EVs per 70 kg patient mass, and (d) 2.0×10⁹ EVs per 70 kg patient mass, and the vascular injury is and intensified microvascular stroke pathology.

An embodiment of the presently disclosed invention is related to cells and a method of creating enhanced human Placenta Mesenchymal Stem Cells (hPMSCs) comprising culturing the hPMSCs with cholesterol for one of between 24 and 96 hours and between 48 and 72 hours. According to a further embodiment the cholesterol is between a 1:100 dilution and 1:100 dilution, preferably between a 1:200 dilution and 1:500 dilution, and most preferably a 1:250 dilution. According to a further embodiment a lipid emulsion is further included in the hPMSCs culture. According to a further embodiment the lipid emulsion is a CD lipid concentrate at between a 1:25 and 1:500 dilution, preferably a 1:50 and 1:200 dilution, and most preferably a 1:100 dilution. According to a further embodiment the stem cells are in a frozen state and stored in a cell number designed for stroke therapy administration. According to a further embodiment the stem cells are stored in units of one of 10 million, 20 million, 50 million, 100 million, 200 million, and 500 million. According to a further embodiment ACE2 expression is increased using an ACE2 activator. According to a further embodiment the ACE2 activator is diminazene.

An embodiment of the presently disclosed invention is related to exosomes and a method of creating enhanced exosomes from hPMSCs comprising culturing the hPMSCs with cholesterol for one of between 24 and 96 hours and between 48 and 72 hours, and then separating the enhanced exosomes from the hPMSCs. According to a further embodiment, media containing the hPMSCs and exosomes are centrifuged. According to a further embodiment a resulting supernatant is transferred to microcentrifuge tubes and re-centrifuged at an increased gravity to pellet extracellular vesicles (EVs) that include exosomes. According to a further embodiment, a second supernatant was aspirated, and EVs pellet was washed twice by centrifugation using 4° C. PBS/1 mM phenylmethylsulfonyl fluoride (PMSF) and pelleted a final time at 20,800 g for 15 min (4° C.), and supernatants were aspirated. According to a further embodiment, exosomes are isolated from the EVs by one of two-step differential centrifugation step, use of Annexin V-coated magnetic beads, sucrose gradient centrifugation, immunoisolation, ExoMir® filtration technologies, ExoQuick® precipitation technologies, size filtration, and/or ultracentrifugation. According to a further embodiment the cholesterol is between a 1:100 dilution and 1:100 dilution, preferably between a 1:200 dilution and 1:500 dilution, and most preferably a 1:250 dilution. According to a further embodiment a lipid emulsion is further included in the hPMSCs culture. According to a further embodiment the lipid emulsion is a CD lipid concentrate at between a 1:25 and 1:500 dilution, preferably a 1:50 and 1:200 dilution, and most preferably a 1:100 dilution. According to a further embodiment the exosomes are in a frozen state and stored in an amount designed for stroke therapy administration. According to a further embodiment, the amount of exosomes is equivalent to an amount of exosomes that are separated from of one of 10 million, 20 million, 50 million, 100 million, 200 million, and 500 million hPMSCs.

An embodiment of the presently disclosed invention is related to microparticles and a method of creating enhanced microparticles from hPMSCs comprising culturing the hPMSCs with cholesterol for one of between 24 and 96 hours and between 48 and 72 hours, and then separating the enhanced microparticles from the hPMSCs. According to a further embodiment, media containing the hPMSCs and microparticles are centrifuged. According to a further embodiment a resulting supernatant is transferred to microcentrifuge tubes and re-centrifuged at an increased gravity to pellet EVs that include microparticles. According to a further embodiment, a second supernatant was aspirated, and EVs pellet was washed twice by centrifugation using 4° C. PBS/1 mM phenylmethylsulfonyl fluoride (PMSF) and pelleted a final time at 20,800 g for 15 min (4° C.), and supernatants were aspirated. According to a further embodiment microparticles are isolated from the EVs by one of two-step differential centrifugation step, use of Annexin V-coated magnetic beads, sucrose gradient centrifugation, immunoisolation, ExoMir® filtration technologies, ExoQuick® precipitation technologies, size filtration, and/or ultracentrifugation. According to a further embodiment the cholesterol is between a 1:100 dilution and 1:100 dilution, preferably between a 1:200 dilution and 1:500 dilution, and most preferably a 1:250 dilution. According to a further embodiment a lipid emulsion is further included in the hPMSCs culture. According to a further embodiment the lipid emulsion is a CD lipid concentrate at between a 1:25 and 1:500 dilution, preferably a 1:50 and 1:200 dilution, and most preferably a 1:100 dilution. According to a further embodiment the microparticles are in a frozen state and stored in an amount designed for stroke therapy administration. According to a further embodiment the amount of microparticles is equivalent to an amount of microparticles that are separated from of one of 10 million, 20 million, 50 million, 100 million, 200 million, and 500 million hPMSCs.

An embodiment of the presently disclosed invention is related to EVs including exosomes and microparticles and a method of creating enhanced EVs from hPMSCs comprising culturing the hPMSCs with cholesterol for one of between 24 and 96 hours and between 48 and 72 hours, and then separating the enhanced EVs from the hPMSCs. According to a further embodiment, media containing the hPMSCs and EVs are centrifuged. According to a further embodiment a resulting supernatant is transferred to microcentrifuge tubes and re-centrifuged at an increased gravity to pellet EVs. According to a further embodiment, a second supernatant was aspirated, and EVs pellet was washed twice by centrifugation using 4° C. PBS/1 mM phenylmethylsulfonyl fluoride (PMSF) and pelleted a final time at 20,800 g for 15 min (4° C.), and supernatants were aspirated. According to a further embodiment the cholesterol is between a 1:100 dilution and 1:100 dilution, preferably between a 1:200 dilution and 1:500 dilution, and most preferably a 1:250 dilution. According to a further embodiment a lipid emulsion is further included in the hPMSCs culture. According to a further embodiment the lipid emulsion is a CD lipid concentrate at between a 1:25 and 1:500 dilution, preferably a 1:50 and 1:200 dilution, and most preferably a 1:100 dilution. According to a further embodiment the EVs are in a frozen state and stored in an amount designed for stroke therapy administration. According to a further embodiment the amount of EVs is equivalent to an amount of EVs that are separated from of one of 10 million, 20 million, 50 million, 100 million, 200 million, and 500 million hPMSCs.

An embodiment of the presently disclosed invention further relates to methods and therapeutics comprising a plurality of hPMSCs, wherein the hPMSCs were cultured with cholesterol, such that the hPMSCs have an increased number of extracellular vesicles. According to a further embodiment the hPMSCs have one of 2, 3, 4, 5, and 6 times the number of extracellular vesicles as a hPMSC that is cultured in the absence of cholesterol.

An embodiment of the presently disclosed invention is related to therapeutics and methods of treating a stroke in a patient comprising delivering stem cells into a peritoneum of the patent. According to a further embodiment the stem cells are MSC. According to a further embodiment the stem cells are hPMSC. According to a further embodiment the stroke is one of stroke is one of occlusive, post-occlusive, hemorrhagic, and transient ischemic injury. According to a further embodiment the method further comprises the step of delivering a clot busting compound into a blood stream of the patient. According to a further embodiment the stem cells are delivered into the peritoneum of the patent one of before, coincident with, and after the clot busting compound is introduced into the patient, and wherein the clot busting compound is tissue plasminogen activator. According to a further embodiment the stem cells are delivered via injection. According to a further embodiment a number of stem cells introduced is between one of 1 million and 5 billion, 10 million and 2 billion, and 100 million and 500 million. According to a further embodiment the stem cells are immortalized. According to a further embodiment the stem cells are lentivirally immortalized. According to a further embodiment the stem cells are delivered one of before the stroke occurs, coincident with the stroke occurring, within 1 hour of the stroke occurring, and between an earliest of 0.5, 1, 4, 12, and 24 hours of the stroke occurring and a latest of 1, 4, 12, and 24 hours of the stroke occurring. According to a further embodiment the stem cells have an increased number of extracellular vesicles. According to a further embodiment the stem cells have been cultured with a sterol. According to a further embodiment the sterol is cholesterol. According to a further embodiment the stem cells are cultured with cholesterol for one of between 24 and 96 hours and between 48 and 72 hours. According to a further embodiment a lipid emulsion is further included in the stem cells are additionally culture. According to a further embodiment the stem cells are in a frozen state and stored in a cell number designed for stroke therapy administration. According to a further embodiment the stem cells are stored in units of one of 10 million, 20 million, 50 million, 100 million, 200 million, and 500 million.

An embodiment of the presently disclosed invention further relates to methods and therapeutics comprising a plurality of hPMSCs, wherein the hPMSCs were cultured with cholesterol, such that the hPMSCs have an increased number of extracellular vesicles. According to a further embodiment the hPMSCs have one of 2, 3, 4, 5, and 6 times the number of extracellular vesicles as a hPMSC that is cultured in the absence of cholesterol.

An embodiment of the presently disclosed invention further relates to therapeutics and methods of treating a stroke in a patient comprising administering a therapy to cause reperfusion in the patient, injecting immortalized hPMSC into a peritoneum of the patent substantially at a same time as the reperfusion therapy is administered wherein the stroke is one of stroke is one of occlusive, post-occlusive, hemorrhagic, and transient ischemic injury, a number of stem cells introduced is between one of 1 million and 5 billion, 10 million and 2 billion, and 100 million and 500 million, the stem cells have been cultured with cholesterol and a lipid emulsion for between 24 and 96 hours.

The inventors describe the use of intraperitoneal stem cells as a safe alternative which provides nearly complete protection against acute stroke injury in the occlusive model of stroke.

An embodiment of the presently disclosed invention relates to the use of immortalized placental stem cells (IPSC) and/or extracellular vesicles derived therefrom to enhance therapeutic recovery from tissue damage and ischemia-reperfusion injury and delayed organ function.

An embodiment of the presently disclosed invention relates to a novel, more effective and safer approach to use human placenta mesenchymal stem cells as protective agents in stroke models and in clinical stroke as well as ischemia reperfusion injury. A further embodiment of the invention is to use frozen stem cells which are thawed on demand to provide a more easily accessible option for patients undergoing stroke therapy. This treatment is intended to provide cell therapy beyond the 4 hour window now used for stroke. This protection is mediated at least in part by stem cell-derived exosome/microparticles and their expression of angiotensin converting enzyme 2. The numbers and efficacy of these particles may be increased by supplementing stem cells with cholesterol supplements prior to stem cell or exosome administration and/or by increasing ACE2 expression using diminazene, a described ACE2 activator.

An embodiment of the presently disclosed invention uses lentivirally immortalized human placenta derived, autologous or other origin stem cell as a therapeutic approach to maintain blood flow and preserve tissue integrity. This invention involves the creation of sufficient cells and/or extracellular vesicles which can be frozen for later therapeutic administration. Additionally, these cells enable the creation of much greater recovery of extracellular vesicles to permit administered intravenous as well as intraperitoneal administration. Importantly, the process makes it possible to obtain large enough numbers of cells for a clinical ‘dose’ which can provide protection against stroke injury (tested in the middle cerebral artery occlusion model of stroke (MCAO)). We found that administration of 500,000 stem cells into the peritoneum was significantly protective of tissue structure and neurological outcomes in stroke models. However, scaling this to human therapy could require 1-2 billion cells which could be difficult to obtain based on typical cell proliferation rates. However, these IPSC grow extremely rapidly, provide equivalent protection as non-transformed cells and represent a novel therapeutic. Similarly, vesicles derived from these cells are also effective and therapeutic and may have improved activity towards maintenance/restoration of blood flow and tissue function.

An embodiment of the presently disclosed invention would be used to treat individuals who are seen at emergency departments for thrombotic strokes, particularly when tissue plasminogen activator (tPA) is administered. This treatment when given after a stroke would prevent the progressive brain tissue destruction, loss of function and behavioral and motor disturbances which often accompany stroke even when tPA is administered.

According to an embodiment of the presently disclosed invention, a cell-based exosome ‘product’ would be provided which would ultimately be used after every tissue plasminogen treatment for stroke ‘clot-busting’. This product could be banked and shipped and used at later dates and allow for much greater quality control. It is much safer than the intravenous use of stem cells.

An embodiment of the presently disclosed invention relates to ischemic stroke injury and therapy. A disclosed method overcomes the relatively dangerous current use of stem cells in an occlusive crisis in the brain vasculature. Because ‘clot busting’ anticipates the removal of a blood clot using tissue plasminogen activator (TPA), intravenous stem cells present potential risks. By comparison intraperitoneal stem cells (administered into the abdomen) are much safer and highly effective, potentially almost completely eliminating stroke injury in the middle cerebral artery occlusion model.

An embodiment of the presently disclosed invention involves administering human placenta stem cells by an intraperitoneal route prior to or following stroke onset and/or prior to, concurrent with, or following the administration of “clot busting” therapies.

An embodiment of the presently disclosed includes the use of extracellular vesicles/microparticles, which are prepared from normal or cholesterol/lipid treated/enhanced stem cells, as therapies for stroke, which are cryostorable and which preserve blood flow, neurological function and tissue structure in the post-stroke affected brain. The extracellular vesicles/microparticles may be isolated from the stem cells and administered to the patient independent of the stem cells.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIG. 1 is a schematic representation of how ACE2 contributes to hPMSC-induced protection against ischemic injury through MasR pathway.

FIGS. 2A-2C show that ACE2 contributes to the hPMSCs-based protection in the MCAO model. FIG. 2A shows western blot for expression of ACE2 in hPMSCs and hBMEC-D3 cells. Quantification analysis of ACE2 expression normalized to β-tubulin protein expression showed significantly higher levels of ACE2 in the hPMSCs compared to hBMEC-D3 cells (*p=0.04). FIG. 2B shows neurological scores calculated based on 24 scale evaluation tests. Significant differences in neurological scores were seen between MCAO (n=8) and sham groups (****p<0.0001). hPMSC treatment (n=8) significantly improved neurological scores vs MCAO group (****p<0.0001). One-way ANOVA analysis showed no significant differences in neurological functions between the MCAO group and MCAO mice treated with MLN-hPMSC (NS, p=0.74, n=6). Neurological scores were significantly reduced in MLN-hPMSC treated MCAO vs hPMSC-treated MCAO mice (****p<0.0001). FIG. 2C shows infarcted areas were assessed by TTC staining. Significant increase in infarcted volume was observed in MCAO+MLN-treated hPMSC vs MCAO+hPMSC mice (****p<0.0001). No significant differences of infarction were observed between MCAO+MLN-treated hPMSC mice vs MCAO (NS, p=0.89). In all graphs, data represent means±SEM.

FIGS. 3A-3D show ACE2 contributes to the hPMSCs-based preservation of blood flow in the MCAO model. FIG. 3A shows total cerebral blood flow measured using Laser Speckle imaging. ‘Perfusion’ reflects total cerebral flow signal measured in selected tissue areas. Measurements are expressed as perfusion units (PU), using a fixed scale (arbitrary units). Significant reduction was observed in the perfusion of MCAO+MLN-treated hPMSCs (n=7) compared to MCAO+hPMSC (n=7) (****p<0.0001). One-way ANOVA analysis showed no significant differences in the cerebral blood flow between MCAO+MLN-treated hPMSC and MCAO (n=10) groups (NS, p=0.1). Cerebral perfusion in each pair of ipsilateral (FIG. 3B) and contralateral (FIG. 3C) brain hemispheres were normalized to the average sham total CBF as the reference point. A significant decrease in normalized CBF was observed in the ipsilateral hemisphere of both MCAO (78%; ****p<0.0001) and MLN-hPMSC treated MCAO (61%; ****p<0.0001) groups was observed compared to the sham group. A significant decrease in normalized CBF in the contralateral hemisphere of both MCAO (60%; ****p<0.0001) and MLN-hPMSC treated MCAO (40%; ***p=0.0004) groups was observed compared to the sham group. Significant differences were observed in both ipsilateral (****p<0.0001) and contralateral (*p=0.02) hemispheres of MCAO+MLN-hPMSCs group compared to MCAO+hPMSCs mice. As shown in Fig. D, no significant differences were observed in the relative distribution of normalized CBF of contralateral vs ipsilateral hemisphere of both sham (NS; p>0.99) and hPMSC-treated MCAO groups (NS; p=0.13). Two-way ANOVA analysis revealed significant differences between contralateral and ipsilateral perfusion of both MCAO (*p=0.04) and MLN-hPMSC treated MCAO (*p=0.03) mice. All graph data show the means±SEM.

FIGS. 4A-4D show downregulation of ACE2 expression eliminates hPMSCs-induced protection in the MCAO model. FIG. 4A shows representative image of hPMSCs transfected with lentivirus-shACE2-GFP. Scale bars, 100 μm. FIG. 4B shows Western blot analysis showed significant reduction in protein expression of ACE2 in lentiviral-GFP⁺-shACE2-transfected hPMSCs compared to the control hPMSCs (***p=0.002). FIG. 4C shows TTC staining of brain slices revealed a significant increase in infarcted volume in the ipsilateral hemisphere of MCAO+shACE2-hPMSCs (n=6) versus MCAO+hPMSC (n=6) mice (****p<0.0001). No significant differences of infarction were observed between MCAO+shACE2-hPMSCs mice versus MCAO (n=6) (NS, p=0.06). FIG. 4D shows neurological scores were significantly reduced in shACE2-hPMSC treated MCAO (n=6) vs hPMSC-treated MCAO (n=6) mice (****p<0.0001). One-way ANOVA analysis showed no significant differences in neurological functions between MCAO group (n=6) and MCAO mice treated with shACE2-hPMSC (NS, p>0.99). In all graphs, data represent means±SEM.

FIGS. 5A-5D show downregulation of ACE2 expression abolishes hPMSCs-based preservation of blood flow in the MCAO model. FIG. 5A shows Laser Speckle imaging revealed a significant reduction in the cerebral blood flow of MCAO+shACE2-hPMSCs compared to MCAO+hPMSC (***p=0.0005). FIG. 5B shows significant decrease in blood flow into the ipsilateral hemisphere of MCAO (****p<0.0001, n=7) and MCAO+shACE2-hPMSCs (****p<0.0001, n=6) compared to hPMSC-treated MCAO mice (n=6). FIG. 5C shows significant decrease in blood flow into the contralateral hemisphere of MCAO (****p<0.0001) and MCAO+shACE2-hPMSCs (*p=0.02) brains was detected compared to the MCAO+hPMSC groups. FIG. 5D shows two-way ANOVA analysis revealed significant changes between ipsilateral and contralateral perfusion in MCAO (*p=0.01) and MCAO+shACE2-hPMSCs (****p<0.0001) groups. No significant differences were detected in ipsilateral and contralateral perfusion of sham (NS, p>0.99) and MCAO+hPMSC (NS, p=0.053). All graph data show the means±SEM.

FIGS. 6A-6B show hPMSCs-derived ACE2 protects against ischemic injury independent of AT2R pathway. The AT2R antagonist, PD 123319 (10 mg/kg i.v.), injected into C57BL/6 mice 1 h before MCAO surgery to block the AT2R. FIG. 6A shows TTC staining of brain slices. No significant differences were found in infarct sizes of PD-pretreated MCAO and untreated MCAO mice (NS, p=0.36; n=6). IP injection of hPMSC (5×10⁵ cells in 500 ml HBSS) significantly reduced the infarction in PD-pretreated MCAO mice (****p<0.0001; n=6). FIG. 6B shows neurological scores were significantly improved in hPMSC-injected PD-MCAO group vs untreated PD-MCAO mice (***p=0.0002; n=6). One-way ANOVA analysis showed no significant differences in neurological functions between MCAO and PD-pretreated MCAO mice (NS, p=0.39). In all graphs, data represent means±SEM.

FIGS. 7A-7 C show hPMSCs-derived ACE2 restores blood perfusion in MCAO mice independent of AT2R pathway. FIG. 7A shows Laser Speckle measurement of brain perfusion showed no significant differences in total perfusion between PD-pretreated MCAO (n=5) and MCAO (n=6) mice (NS, p>0.99). Significant increases in blood flow into the brain of PD-pretreated MCAO+hPMSCs (n=5) mice were observed compared to PD-pretreated MCAO (**p=0.001). FIG. 7B shows significant elevation of blood perfusion observed in the ipsilateral hemisphere of PD-MCAO+hPMSC mice compared to PD-MCAO group (**p=0.001). FIG. 7C shows significant increase of blood flow into the contralateral hemisphere of hPMSC-treated PD-MCAO (*p=0.01) observed in comparison with untreated PD-MCAO mice. There were no significant changes in ipsilateral (NS, p>0.99) and contralateral (NS, p=79) perfusion of MCAO vs PD-MCAO mice. Two-tailed Student's t-test analysis did not show any significant differences in blood flow between the ipsilateral (NS, p=0.55) and contralateral (NS, p=0.55) hemispheres of PD-pretreated MCAO, regardless of hPMSCs treatment. FIG. 7D shows a two-way ANOVA analysis revealed significant disturbances in blood perfusion between ipsilateral and contralateral hemispheres of MCAO (*p=0.01) and PD-MCAO (*p=0.03) groups. No significant differences were detected between ipsilateral and contralateral perfusion of sham (NS, p>0.99), MCAO+hPMSC (NS, p=0.75), and PD-MCAO+hPMSC (NS, p=0.58). All graph data show the means±SEM.

FIGS. 8A-8B show masR pathway contributes to the ACE2-mediated protection in the MCAO model. Protective potential of hPMSC via masR pathway was tested in the MCAO mice pretreated with masR antagonist (A779; 80 mg/kg i.p). No significant differences were observed in the (FIG. 8A) infarction area (TTC staining; NS, p=0.36) and (FIG. 8B) neurological scores (NS, p=0.52) of the MCAO+A779 treated IP with hPMSC (n=4) compared to untreated MCAO+A779 (n=4) group. All graph data show the means±SEM.

FIGS. 9A-9D show masR pathway contributes to the ACE2-mediated blood flow preservation in the MCAO model. Laser Speckle imaging did not reveal any significant changes in (FIG. 9A) total (NS, p=0.79), (FIG. 9B) ipsilateral (NS, p=0.57), and (FIG. 9C) contralateral (NS, p=0.47) blood perfusion of the MCAO+A779 treated IP with hPMSC (n=4) compared to untreated MCAO+A779 (n=4) group. FIG. 9D shows a two-way ANOVA analysis showed significant disturbances in blood perfusion between ipsilateral and contralateral hemispheres of MCAO (*p=0.01), A779-MCAO (*p=0.03), and hPMSC-treated A779-MCAO (***p=0.0008) groups. All graph data show the means±SEM.

FIGS. 10A-10L show administration of hPMSC protects against ischemic injury in the MCAO stroke model. FIG. 10A shows the area of infarction (‘white’ area) in each brain slice was visualized using TTC staining and measured using Image-J (NIH). Unstained (‘white’) regions in brain slices were combined to generate a composite tissue injury score for each brain. Each point represents one animal (sham (n=7), MCAO (n=6), MCAO+hPMSC (n=8)). Compared to sham, there was a significant increase in infarct size in MCAO mice (****p<0.0001, one-way ANOVA). By comparison infarct size in hPMSC-treated MCAO mice was significantly reduced (****p<0.0001, Student's t-test). FIG. 10B shows total cerebral perfusion measured using Laser Speckle imaging. ‘Perfusion’ reflects total cerebral flow signal measured in selected tissue areas. Measurements are expressed as perfusion units (PU), using a fixed scale (arbitrary units). Significant differences were observed between cerebral perfusion in MCAO (n=6) and sham (n=6) mice (****p<0.0001, one-way ANOVA). hPMSC-treated MCAO (n=7) mice showed significant preservation of perfusion compared to non-treated post-MCAO group (****P<0.0001, Student's t-test). FIGS. 10C and 10D show cerebral perfusion in each pair of ipsilateral (10C) and contralateral (10D) brain hemispheres were normalized to the average sham total CBF as the reference point. A large (83%) decrease in cerebral perfusion of the ipsilateral side of MCAO brain was observed compared to the sham operated group (****p<0.0001, one-way ANOVA). A significant decrease (65%) in cerebral perfusion of the contralateral hemisphere of MCAO brain was observed compared to the sham group (****p<0.0001, one-way ANOVA). hPMSC treatment significantly preserved blood flow normal distribution between the hemispheres (far right bars in FIGS. 10C and 10D); significant differences determined by Student's t-test, ****p<0.0001 and ****p<0.0001 for comparison of hPMSC-treated MCAO mice to untreated MCAO ipsilateral/contralateral hemispheres, respectively. FIG. 10E shows neurological scores 24 h post-reperfusion. Significant differences in neurological scores were seen between untreated MCAO (n=8) and hPMSC-treated MCAO (n=8) to sham (n=8) group (****p<0.0001, one-way ANOVA). There was a significant improvement in neurological score in hPMSC-treated MCAO mice compared to untreated MCAO (****p<0.0001, Student's t-test). FIG. 10F shows neuronal degeneration in MCAO with hPMSC therapy. Neurons (black arrows), degenerating neurons (black arrows head), glial cells (red arrows head), and spongiform regions (red arrows) were visualized using Nissl staining of brain sections in untreated and hPMSC-treated MCAO groups. Two-tailed Student's t-test analysis revealed significant differences in proportions of neurons (*p=0.02, FIG. 10G), degenerating neurons (*p=0.04, FIG. 10H), and glial cell (*p=0.02, FIG. 10I) numbers in hPMSC-treated MCAO brain sections compared to untreated MCAO sections. In all graphs, data represent means±SEM. FIG. 10J shows representative images after immunohistochemistry staining for Iba-1. FIG. 10K shows quantification of numbers of Iba-1⁺ microglia in the border between striatum and cortex (average of 3 different fields) of ipsilateral hemisphere of MCAO and MCAO+hPMSC groups (NS, p=0.5, Student's t-test, n=4 per group). FIG. 10L shows microglial activation in ipsilateral hemispheres of MCAO and MCAO+hPMSC groups was determined by cell body length (**p=0.002, Student's t-test).

FIGS. 11A-11O show hPMSCs maintain blood brain barrier integrity against MCAO in mice. FIG. 11A shows BBB disruption assessed by Evans blue (EB) leakage into the brains of sham (n=4), MCAO (n=6), hPMSC-treated MCAO (n=5) mice 24 h following reperfusion. Significant differences in EB leakage were observed between MCAO versus sham (****p=0.0001, one-way ANOVA) and hPMSC-treated MCAO mice (***p=0.004, Student's t-test). No significant differences were detected between hPMSC-treated MCAO versus sham (NS, p=0.08, one-way ANOVA). FIG. 11B shows schematic of in vitro model of ischemic stress. FIG. 11C shows barrier function of human brain endothelial (hCMEC-D3) monolayers under normoxia and oxygen-glucose deprivation reperfusion (OGDR) condition were measured using biotinylated-gelatin/FITC-avidin evaluation. Significant differences in fluorescence intensity of D3 monolayers were observed under normoxia versus OGDR (****p<0.0001, One-way ANOVA) and OGDR versus OGDR+hPMSC (*p=0.02, Student's t-test). (I to III) Representative images of biotinylated-gelatin FITC-avidin permeability assay of monolayers under (I) normoxic, (II) OGDR, and (III) OGDR+hPMSC conditions. Scale bars, 100 μm (I to III). FIG. 11D show western blots for expression of ZO-1, Claudin-1, Occludin, and β-tubulin in D3 monolayers under normoxia and OGDR (with/without hPMSC). Quantification of ZO-1 (FIG. 11E), claudin1 (FIG. 11F), and occludin (FIG. 11G) expression normalized to β-tubulin protein expression. No significant differences were observed in the expression of ZO-1 (FIG. 11E; p=0.8), claudin1 (FIG. 11F; p=0.76) or occludin (FIG. 11G; p=0.64) in monolayers under OGDR condition versus OGDR+hPMSC group using two-tailed Student's t-test analysis. Immunoblot and quantification of VE-cadherin (FIGS. 11H and 11I) and α-catenin (FIGS. 11L and 11M) protein expression normalized to β-tubulin. Significant differences were found between OGDR versus OGDR+hPMSC (FIG. 11I; **p=0.01 and FIG. 11M; ***p=0.006, Student's t-test). Immunofluorescence staining and quantification of VE-cadherin (FIGS. 11J and 11K) and α-catenin (FIGS. 11N and 11O). Fluorescence intensity of VE-cadherin and α-catenin (green color; FIGS. 11K and 11O, respectively) was normalized to cell numbers (DAPI-stained nuclei, blue color). Significant differences in fluorescent intensity for VE-cadherin (FIG. 11J; **p=0.01, Student's t-test) and a-catenin (FIG. 11N; **p=0.01, Student's t-test) were observed in hPMSC-treated monolayers under OGDR. Scale bars, 100 μm (FIGS. 11K and 11O).

FIGS. 12A-12F show hPMSC-released EVs contribute to SC-based protection in MCAO. In FIG. 12A, flow cytometry was used to evaluate numbers of extracellular vesicles (EVs) released from hPMSC treated with or without (10 mM) methyl-beta cyclodextrin (MβCD) for 2 hours. Mann Whitney U-test analysis revealed significant differences in numbers of EVs released from hPMSC compared to MβCD-treated hPMSC (**p=0.03). In FIG. 12B, infarcted areas were assessed by TTC staining. Significant differences of infarction were measured between MCAO (n=6) mice versus MCAO+hPMSC (n=8) (****p<0.0001) and MCAO+MβCD-treated hPMSC (n=8) (NS, p>0.99) using one-way ANOVA. Significant increases in the infarcted area were observed in MCAO+MβCD-treated hPMSC vs. MCAO+hPMSC mice (****p=0.0001, Student's t-test). In FIG. 12C, neurological scores of MβCD-treated hPMSC (n=6) mice were comparable to MCAO (n=9) group (NS, p=0.54, one-way ANOVA). Significant differences of neurological scores were observed in MCAO+hPMSC mice versus MCAO group (****p<0.0001; one-way ANOVA) and MβCD-treated hPMSC mice (****p=0.0001; Student's t-test). In FIG. 12D, significant differences in brain perfusion of MCAO+hPMSC (n=6) mice subjected to MCAO (n=6) (****p<0.0001, one-way ANOVA) and those given MβCD-treated hPMSC (n=6) (****p<0.0001, Student's t-test) group were measured. No significant differences were detected in MCAO group compared to MβCD-treated hPMSC group (NS, p>0.99, one-way ANOVA). In FIG. 12E, significant decreases in blood flow into the ipsilateral hemisphere of MCAO (****p<0.0001, one-way ANOVA) and MCAO+MβCD-treated hPMSC (****p<0.0001, Student's t-test) treated mice were compared to hPMSC-treated MCAO groups. Changes in the ipsilateral perfusion of MCAO and MCAO+MβCD-treated hPMSC groups were not significant (NS, p>0.99, one-way ANOVA). In FIG. 12F, significant decreases in blood flow into the contralateral hemisphere of MCAO (****p=0.0004, one-way ANOVA) and MCAO+MβCD-treated hPMSC (***p=0.001, Student's t-test) brains was detected compared to MCAO+hPMSC groups. No significant differences of contralateral perfusion were observed between MCAO and MCAO+MβCD-treated hPMSC mice (NS, p>0.99, one-way ANOVA). All graph data show the means±SEM.

FIGS. 13A-13G show cholesterol/lipid supplementation enhances the protective capacity of hPMSCs in MCAO. In FIG. 13A, cholesterol-lipid content of hPMSCs was evaluated using Oil Red O staining which stains lipid droplets and intracellular cholesterol. No significant changes in intracellular cholesterol/lipid were observed (NS, p=0.7, Student's t-test). In FIG. 13B, numbers of EVs released from hPMSCs and cholesterol-treated hPMSCs were calculated using flow cytometric analysis. Significant increases in numbers of EVs released from cholesterol-treated hPMSCs were found compared to untreated hPMSCs (**p=0.02, Mann Whitney U test). Protective potential of 1×10⁵ hPMSCs and cholesterol-treated hPMSCs were compared in MCAO. Significant differences of (FIG. 13C) infarcted area (TTC staining; NS, p=0.23 and ****p<0.0001), (FIG. 13D) total cerebral blood flow (NS, p=0.59 and ****p=0.0002), and (FIG. 13G) neurological scores (NS, p=0.06 and ****p<0.0001) were measured using one-way ANOVA to compare MCAO (n=5) with MCAO+hPMSC (n=6) and MCAO+Chl-hPMSC (n=6) groups. In FIG. 13E, ipsilateral perfusion improved in MCAO+Chl-hPMSC mice compared to MCAO (****p<0.0001, one-way ANOVA) and MCAO+hPMSC (****p<0.0001, Student's t-test) groups. Significant decreases in perfusion into ipsilateral hemispheres of both MCAO (****p<0.0001) and MCAO+hPMSC (****p<0.0001) groups versus sham mice was observed (one-way ANOVA). In FIG. 13F, significantly decreased blood flow in contralateral hemispheres in MCAO (****p<0.0001) and MCAO+hPMSC (****p<0.0001) was observed (versus sham). Redistribution of contralateral perfusion in MCAO+Chl-hPMSC mice was comparable to sham (NS, p=0.43, one-way ANOVA) groups. No significant differences were detected in ipsilateral (p>0.99) or contralateral (p=0.19) hemispheres of MCAO mice compared to MCAO+hPMSC mice (by one-way ANOVA.) All graph data show means±SEM.

FIGS. 14A-14F show IV injection of EVs derived from Chl-treated hPMSCs is protective in MCAO. In FIG. 14A, flow cytometric analysis showed that Chl-treatment significantly reduced phosphatidylserine (PS) expression (decreased Annexin-V⁺) on EVs (**p=0.03, Mann Whitney U test). Protective potency of IV injection of Chl-treated hPMSC-EVs (2×10⁶ EVs in 100 μl HBSS) was evaluated in MCAO. Significant differences in infarcted area (FIG. 14B; ****p<0.0001), total cerebral perfusion (FIG. 14C; ****p<0.0001), ipsilateral perfusion (FIG. 14D; ****p<0.0001), contralateral perfusion (FIG. 14E; ****p<0.0001) and neurological scores (FIG. 14F; ****p<0.0001) of MCAO+Chl-treated EVs (n=7) and untreated MCAO (n=5) groups were identified using Student's t-test analysis. The above parameters: FIG. 14C; total perfusion (p=0.49), FIG. 14D; ipsilateral perfusion (p=0.14), FIG. 14E; contralateral perfusion (p=0.55), FIG. 14F; neurological scores (p=0.07)) in MCAO+Chl-treated EVs mice were comparable to the sham group (one-way ANOVA). All graphed data show means±SEM.

FIGS. 15A-15D show MCAO model of Stroke. In FIG. 15A, there were no significant differences in infarction between MCAO (n=4) and sham group (n=4) at time point of 4 hours following reperfusion (NS, Student t-test analysis). In FIG. 15B, significant differences of infarcted area (TTC staining) in MCAO mice (n=10) versus sham group (n=7) was detected 24 hours after reperfusion using Student t-test analysis, ****P<0.0001. In FIG. 15C, significant differences of total perfusion into the brain (Laser Speckle Imaging) of MCAO mice (n=4) versus sham group (n=4) was detected 24 hours after reperfusion using Student t-test analysis, ****P=0.0008. In FIG. 15D, significant differences of neurological scores in MCAO mice compared to sham group was detected 24 hours after reperfusion using Student t-test analysis, ****P<0.0001.

FIGS. 16A-16G show characteristics of hPMSC. In FIG. 16A, calcein AM staining showed spindle shape of hPMSCs in culture. Scale bar, 100 mm. Fluorescence-activated cell sorting (FACS) analysis detected the expression of CD73 (FIG. 16B) and CD90 (FIG. 16C) on hPMSC. Immunostaining of hPMSC was positive for markers of CD44 (FIG. 16D) and Oct3/4 (FIG. 16E). DAPI used for nuclear staining (FIGS. 16A, 16D, and 16E). In FIGS. 16F and 16G, FACS analysis showed negative expression of HLA-DR and CD34 markers, respectively, on hPMSC.

FIG. 17 shows a comparison of blood flow between contralateral and ipsilateral hemispheres. Laser Speckle imaging analysis showed no significant differences in blood perfusion of contralateral (51.71%) versus ipsilateral (49.17%) hemisphere of sham animals (NS; p>0.99; two-way ANOVA). Two-way ANOVA analysis revealed significant differences between contralateral perfusion (20%) compared to ipsilateral (6%) site of MCAO group (p=0.04). In comparison, there was no significant differences between contralateral (35%) and ipsilateral (29%) hemispheres of MCAO mice treated with hPMSC (NS; p=0.5).

FIGS. 18A-18F show expression of tight/adherens junctional proteins under OGDR condition. Protein expression of tight junction proteins (ZO-1, claudin-1, occludin) and adherens junction proteins (VE-cadherin and a-catenin) of hCMEC-D3 monolayers under oxygen glucose deprivation reperfusion (OGDR) condition at different time points of 6, 12, and 16 hours was measured by western blot analysis. Statistical differences were determined by two-way ANOVA and Sidak's multiple comparisons tests for comparisons of OGDR condition at 6, 12, and 12 time points. For quantification, expression of each protein normalized to protein expression of β-tubulin. Significant differences in protein expression of each protein under OGDR compared to normoxia at different time points as follow: FIG. 18A shows ZO-1 (6 h; p=0.99, 12 h; p=0.03, 16 h; p=0.05); FIG. 18B shows claudin-1 (6 h; p=0.11, 12 h; p=0.003, 16 h; p=0.02); FIG. 18C shows occludin (6 h; p=0.14, 12 h; p=0.002, 16 h; p=0.0.005); FIG. 18D shows VE-cadherin (6 h; p=0.50, 12 h; p=0.04, 16 h; p=0.05); FIG. 18E shows a-catenin (6 h; p=0.96, 12 h; p=0.02, 16 h; p=0.02). FIG. 18F shows the representative western blots.

FIGS. 19A-19F show expression of tight/adherens junctional proteins of hCMEC-D3 monolayers co-cultured with hPMSCs under normoxia. Protein expression of tight junctional proteins (ZO-1, claudin-1, occludin) and adherens junctional proteins (VE-cadherin and a-catenin) of hCMEC-D3 monolayers under normal (normoxia) condition when co-cultured (contact independently) with hPMSC for 24 hours and 48 hours compared to the correspondent control using western blot analysis. Statistical differences were measured by two-way ANOVA and Sidak's multiple comparisons tests. For quantification, expression of each protein normalized to protein expression of β-tubulin. No significant differences were detected in protein expression of each protein in hCMEC-D3 monolayers±co-cultured hPMSC (24, 48 hours) as shown here: FIG. 19A shows ZO-1 (24 h; p=0.96, 48 h; p=0.64); FIG. 19B shows claudin-1 (24 h; p=0.98, 48 h; p=0.78); FIG. 19C shows occludin (24 h; p=0.67, 48 h; p=0.78); FIG. 19D shows VE-cadherin (24 h; p=0.46, 48 h; p=0.69); FIG. 19E shows a-catenin (24 h; p=0.92, 48 h; p=0.28). FIG. 19 shows the representative western blots.

FIGS. 20A-20C show hPMSCs do not integrate into the brain after ischemic injury. In FIG. 20A, to track intraperitoneally injected hPMSCs in the MCAO model, hPMSCs were labelled using CytoID red long-term cell tracer kit. In FIG. 20B, blood was collected at time points of 2, 6, and 24 hours after injection, and red-labelled cells were counted by a Nikon video imaging system Eclipse E600FN; using a 20× objective lens. Scale bars, 100 μm. In FIG. 20C, IF staining of hPMSC with anti-human nuclear (Hu-Nu; green) antibody and mouse vascular endothelial cells with anti-mouse CD31 antibody (red); DAPI was used to stain the nuclear of the cells. There was no hPMSC localized in the brain (no green signal was detected). Scale bars, 100 μm.

FIGS. 21A-21C show evaluation of MbCD-treated hPMSCs viability. In FIG. 21A, binocular microscope imaging (20×) did not show any morphological changes between hPMSCs (left panel) and MbCD-treated hPMSCs (right panel). Scale bars, 50 mm. In FIG. 21B, trypan blue test of viability. No significant differences were detected between %viability of hPMSCs and MbCD-treated hPMSCs using Student t-test analysis (NS; p=0.28). In FIG. 21C, MTT assay did not show any significant differences between hPMSCs and MbCD-treated hPMSCs (NS; p=0.98, Student t-test analysis).

FIG. 22 is a table of neurological behavior parameters

FIG. 23 is a table of survival rate of mice after IP or IV injection of hPMSCs or hPMSCs-derived EVs. Row 1: 86% of MCAO mice (non-hPMSCs treated) survived (n=21). Row 2: 12% of normal mice (non-MCAO) survived IV injection of hPMSCs (n=17). Row 3: 87% of MCAO mice survived IP injection of hPMSC (n-15). Row 4: 15% of MCAO mice survived IV injection of PS-positive EV collected from untreated-hPMSC (n=7). Row 5: 80% of MCAO mice survived IV injection of PS-negative EV collected from cholesterol-treated hPMSC (n=10).

FIG. 24 is the original western blots for FIGS. 19A-19F: Expression of junctional proteins in hCMEC-D3 monolayers co-cultured with hPMSC under normoxia after 24 hours.

FIG. 25 is the original western blots for FIGS. 19A-19F: Expression of junctional proteins in hCMEC-D3 monolayers co-cultured with hPMSC under normoxia after 48 hours.

FIG. 26 is the original western blots for FIGS. 18A-18F. Expression of tight/adherens junctional proteins under OGDR condition at 6, 12, and 16 hours.

FIG. 27 is the original western blots for FIGS. 11D, 11H, and 11L: Expression of tight/adherens junction proteins under OGDR condition with/without hPMSC treatment.

FIG. 28 shows administration of immortalized hPMSC protects against ischemic injury in the MCAO stroke model, with the area of infarction (‘white’ area) in each brain slice visualized using TTC staining and measured using Image-J (NIH). Unstained (‘white’) regions in brain slices were combined to generate a composite tissue injury score for each brain. Each point represents one animal.

FIG. 29 shows administration of immortalized hPMSC protects against ischemic injury in the MCAO stroke model, total cerebral perfusion measured using Laser Speckle imaging. ‘Perfusion’ reflects total cerebral flow signal measured in selected tissue areas. Measurements are expressed as perfusion units (PU), using a fixed scale (arbitrary units). Each point represents one animal.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The terms “comprise(s),” “include(s),” “having,” “has,” “can,” “contain(s),” and grammatical equivalents and variants thereof, as used herein, are intended to be open-ended transitional phrases, terms, or words that do not preclude the possibility of additional acts or structures. are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. The singular forms “a,” “and” and “the” include plural references unless the context clearly dictates otherwise. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm.

The embodiments set forth the below represent the necessary information to enable those skilled in the art to practice the invention and illustrate the best mode of practicing the invention. For the measurements listed, embodiments including measurements plus or minus the measurement times 5%, 10%, 20%, 50% and 75% are also contemplated. For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

In addition, the invention does not require that all the advantageous features and all the advantages of any of the embodiments need to be incorporated into every embodiment of the invention.

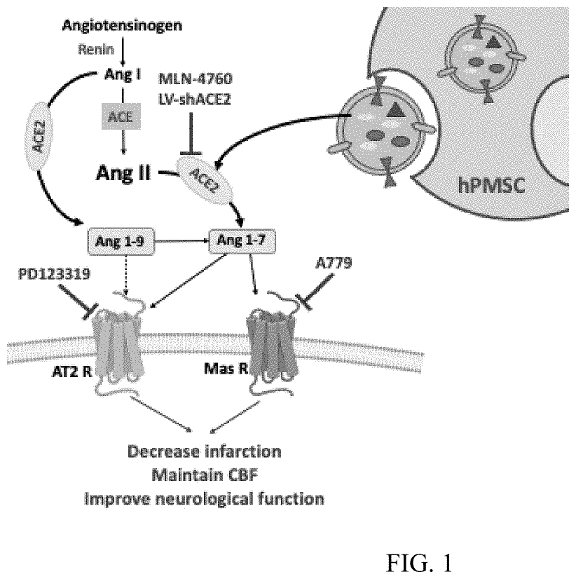

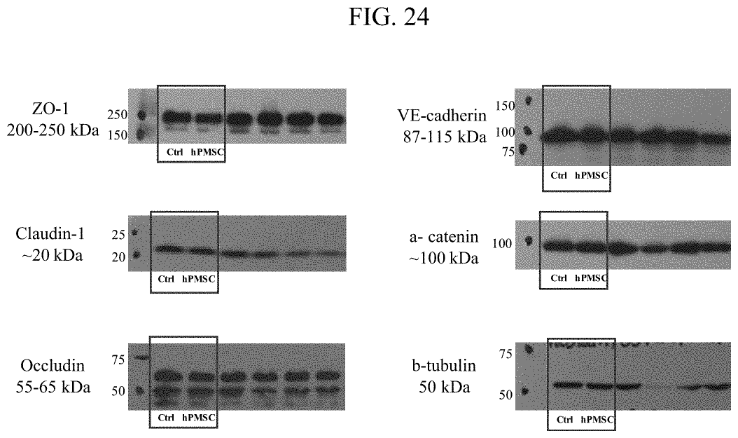

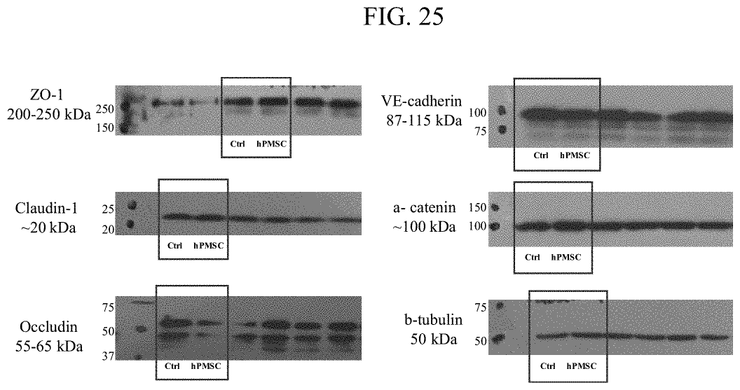

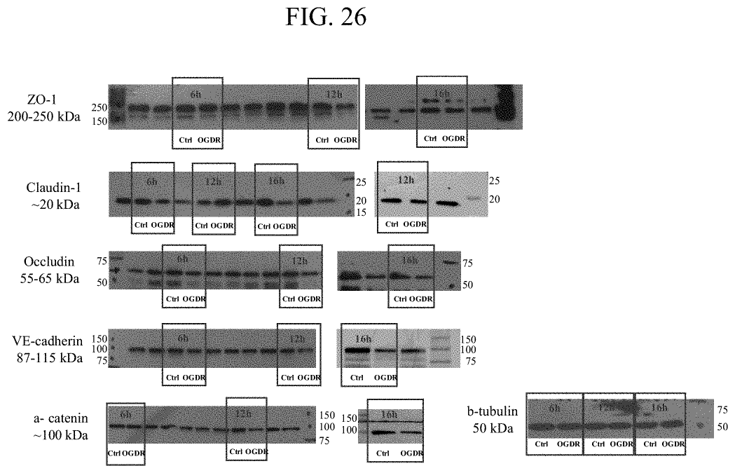

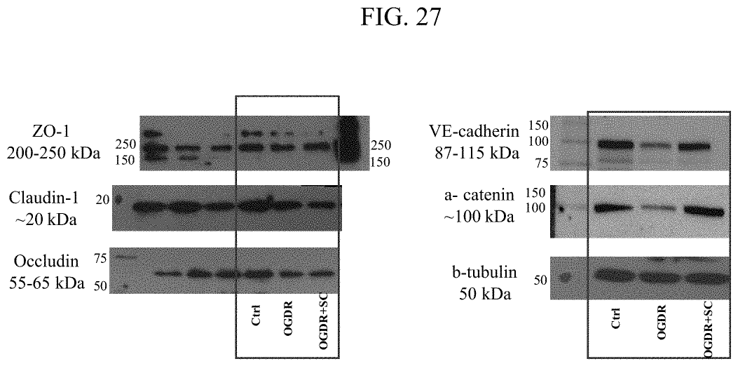

Turning now to FIGS. 1-29, a brief description concerning the various components of the present invention will now be briefly discussed.

Although stem cell therapy for stroke has been previously studied by several groups and in several models, stem cells have still not yet been examined as a therapy in acute stroke for several reasons. The failure for stem cells to be used in stroke treatment may represent, in large part, the significant risk for development of intravascular thrombi when given intravenously in clinical states.

A conceptual advance provided by our current disclosure shows that intraperitoneal administration of immortalized human placenta mesenchymal stem cells (IhPMSC) in MCAO model are powerfully protective against acute stroke injury. Strikingly, these benefits are consistent with paracrine functions of extracellular vesicles derived from hPMSC as biochemical manipulation of membrane cholesterol can positively and negatively alter this protective effect. Our finding also demonstrated that how hPMSC might be safely used in acute therapy for ischemic stroke as this novel approach (intraperitoneal injection of hPMSC) is therapeutically far superior to intravenous stem cell therapy in terms of efficacy. Because this disclosure describes an important and novel set of properties of hPMSC and their derivatives in stem cell therapy, the applications are expected to be rapidly translated as the new standard approach for treating the acute-post ischemic phase in human stroke therapy, potentially saving over 100,000 lives in the United States, and millions of lives in the world every year.

In the US, stroke remains the leading cause of neurologically-mediated disability, and the 3rd leading cause of mortality in adults (1) with stroke incidence and occurrence increasing proportionately with aging in both developed and developing nations. A thromboembolic/ischemic mechanism accounts for up to 85% of stroke with up to 15% hemorrhagic. Ischemic strokes reflect an acute and progressive destruction of neurons, astroglia and oligodendroglia with disruption of the cortical synaptic structure. Maintenance of cerebral blood flow (CBF) is critical for brain function with several protective auto-regulatory mechanisms which ensure adequate perfusion to cerebral arteries under variable conditions. Because of the large cerebral energy demand, it is critical to optimally restore CBF in the acute phase of stroke. A treatment that has been demonstrated to reduce brain damage after stroke is tissue plasminogen activator (t-PA), an enzyme which converts plasminogen to plasmin that dissolves emboli and thrombi, thereby restoring CBF. However, tPA is primarily effective in stroke if administered within 4-5 h of the onset of ischemia. But, paradoxically, the act of restoring local blood perfusion can triggers ischemia/reperfusion injury (IRI) that intensifies stroke severity. Several events contribute to IRI including depletion of energy and oxygen supply, inflammatory infiltration of neutrophils and macrophages into brain tissue, impairment of the blood brain barrier (BBB) and disturbed vasoregulation which lead to irreversible brain injury.

In this disclosure, the inventors tested the therapeutic potential of human placenta-derived mesenchymal stem cells (hPMSCs) in the murine MCAO ischemic stroke model. hPMSCs were chosen because they represent a safe, accessible, abundant, and potentially effective form of SCT. It is also viewed as relatively inexpensive and free of ethical concerns. The inventors then tested immortalized hPMSCs, and found substantially the same efficacy.

The inventors used murine middle cerebral artery occlusion (MCAO) model to monitor changes in infarction size, BBB integrity, and perfusion in the brains of mice with/without hPMSCs and hPMSCs-derived extracellular vesicle (EVs). We found that intraperitoneal (IP) administration of hPMSCs at the beginning of reperfusion (end of 1-hour ischemia) produced remarkable and highly significant preservation of ipsilateral hemispheric blood flow, tissue structure and neurological recovery following MCAO compared to untreated group. Strikingly, these benefits appear to reflect protective effects of EVs released from hPMSCs. Specifically, these benefits seem to be cholesterol-dependent and related to changes in surface presentation of phosphatidylserine (PS). Based on these lines of evidence, we hypothesized that intraperitoneal (IP) administration of hPMSC provided potent protection against stroke-induced infarction, blood brain barrier failure and neurological deficits by maintaining cerebral perfusion for at least 24 h. We further proposed that hPMSC-derived EVs mediate this protection based on: 1) the lack of hPMSC arriving in the bloodstream or brain 2) the ability of cholesterol-lipid supplementation/reduction to influence EV numbers and protection against MCAO and 3) the ability of cholesterol-treated hPMSC to release PS-negative EVs which provide equivalent stroke protection as hPMSC. We further proposed that immortalized hPMSC and EVs from the same would prove to be equally efficacious. The inventors concluded that hPMSC, hPMSC/EVs, and immortalized hPMSC/EVs based stroke therapy represents an important procedure that maintains cerebrovascular perfusion and survival.

### Materials and Methods Part I

Animals: All animal protocols were approved by the LSUHSC-S Institutional Animal Care and Use Committee (IACUC) according to NIH guidelines. We used male C57BL/6J mice (Jackson Laboratories, Bar Harbor, Me.) in all studies at 9-16 weeks of age. Animals were housed in a barrier facility and maintained on a normal diet.

Surgery for MCAO model: Male mice (25-30 g) were anesthetized with ketamine (200 mg/kg i.p.)/xylazine (10 mg/kg i.p.). Once under deep anesthesia, middle cerebral artery occlusion (MCAO) was induced by creating a midline incision at the neck to expose the right carotid bifurcation. The right external carotid artery branch was isolated and ligated, and a micro-clip placed on the common carotid artery. A silicone-coated 6-0-nylon microfilament was introduced into the common carotid artery and the micro-clip released to allow advancement of the filament through the artery until the bulb-tip occluded the origin of the middle cerebral artery (MCA). This filament was left in place for 1 h (ischemia). Reperfusion was initiated by withdrawal of the filament. For sham groups, the right external carotid artery was isolated without further manipulation. The wounds were closed using surgical sutures (6-0) and mice were allowed to recover from anesthesia. Postoperative monitoring of eating, drinking and movement was performed at 4 and 24 h following recovery.

Neurological testing: Neurological outcomes were evaluated at 24 h after reperfusion using a 24-point scale. Briefly, mice were given positive scores (0-3) for each of the following parameters: 5 mins of spontaneous activity, symmetry of movement and forelimbs (outstretching while tail is held), response to vibrissae contact, floor and beam walking, wire cage wall climbing, and reaction to touch on either side of the trunk.

hPMSCs isolation and culture: hPMSCs cells used in this study were isolated. Isolation of placental mesenchymal stem cells (PMSCs) from freshly delivered human placenta was approved by Institutional Review Board (IRB) at Louisiana State University of Health Sciences Center-Shreveport (LSUHSC-S). Briefly, placentas delivered by normal pregnant women were collected immediately after delivery. Villous tissue was separated by sterile dissection from different cotyledons, excluding chorionic and basal plates. After extensive washing with ice-cold phosphate-buffered saline (PBS), villous tissue was digested with trypsin and DNase I in Dulbecco's Modified Eagle's Medium (DMEM) at 37° C. for 90 min. Digested microvillus tissue was collected and cultured in DMEM supplemented with 10% fetal bovine serum (FBS). hPMSCs started to grow in 3-5 days. At ˜80% confluence, the cells were passaged with TrypLE™ Express (Invitrogen, Carlsbad, Calif., USA). hPMSCs were characterized with mesenchymal stem cells markers including positive expression of CD44, CD73 and CD90, and negative expression of CD34 and HLA-DR. Primary isolated hPMSCs also expressed Oct3/4 and were able to differentiate into adipocytes, chondrocytes and osteocytes. hPMSCs were subcultivated at a 1:3 ratio at confluency and passages 3-10 were used in the present study.

IP Injection of hPMSCs: Trypsinized hPMSCs were washed twice with Ca⁺⁺/Mg⁺⁺ free HBSS and centrifuged (1500 RPM, 5 mins, 25° C.). 5×10\hat{ }5 hPMSCs were resuspended in 500 ul HBSS solution without Ca⁺⁺/Mg⁺⁺ and injected intraperitoneally (IP) into MCAO-treated mice at reperfusion.

ACE2 inhibition in hPMSCs: MLN-4760 treatment of hPMSCs. To investigate the contribution of ACE2 in hPMSC-mediated protection, hPMSC were treated with 10 μM MLN-4760 (Sigma Aldrich, USA), which selectively inhibits the activity of ACE2. MLN-treated hPMSC were PBS washed after 48 h treatment and injected (5×10⁵ cells in 500 μL HBSS) into the MCAO mice.

Lentivirus transduction of hPMSCs. Two hours prior to transfection, the medium of HEK293FT at their 90% confluency, was changed to antibiotic free DMEM supplemented with 10% (v/v) FBS. Production of 3^{rd} generation lentivirus was performed combining the transfer vector pLV[shRNA]-EGFP-hACE2 (purchased from VectorBuilder) with packaging plasmid (pMDEL/pRRE, pRSV/REV, and pMD2G), and Lipofectamine3000 enhancer reagent. The mixture was briefly vortexed and incubated at room temperature for 20 min, and then added dropwise to the HEK293FT cells. Flask was agitated gently to distribute the precipitates and then incubated at 37° C., 5% CO2. Four hours late, cell culture media was gently replaced with fresh medium. At 24 hours post-transfection, the medium was replaced with DMEM supplemented with 10% FBS and antibiotics, then incubated at 37° C., 5% CO2. The collection of viral supernatants was made after 48 hours, centrifuged at 1000 rpm for 5 min at 4° C., and passed through a 0.45 mm pore filter to remove cellular debris. Lentivirus was added as droplets to hPMSC (40% confluent) cultured in DMEM supplemented with 10% FBS and antibiotics. After overnight incubation, lentivirus was removed, and fresh media added. Following 48 h, transduction efficiency was determined either by visualizing for expression of fluorescent marker GFP, or western blotting for downregulated expression of ACE2 in hPMSCs.

Laser Speckle measurement of cerebral blood flow: A Perimed Laser Speckle Imaging system (Pericam PSI HR; Sweden) was used to measure cerebral blood perfusion within the brains of the different experimental groups. 24 h after reperfusion, anesthesia was induced and maintained with 3% isoflurane, and mice were placed on a warm pad. The coronal skin was removed, and perfusion recordings accomplished using a high-resolution laser speckle camera (Perimed Laser Speckle Imager) at a working distance of 10 cm. ‘Perfusion’ reflects total cerebral flow signal measured in selected tissue regions of interest. Measurements are expressed as perfusion units (PU), using a fixed scale (arbitrary units). The arbitrary numbers reflecting ‘perfusion’ measured in selected tissue region of interest, then perfusion of either ipsilateral or contralateral was normalized to baseline levels (values obtained from averaged sham total CBF).

TTC staining of infarcted tissue: 24 h after reperfusion, mice were deeply anesthetized with isoflurane and decapitated. The extent and severity of MCAO was evaluated after removal of the brain and staining of brain slices with 2,3,5-Triphenyltetrazolium chloride (TTC; Sigma; USA) to measure infarct size. After dissection, the brain was immersed in cold PBS for 10 m and sliced into 2.0 mm-thick sections using an anatomical slicer. Brain slices were incubated in 2% TTC/PBS for 30 m at 37° C. Areas of infarction in each brain slice were recorded (Nikon 990) and measured using Image-J program (NIH). The infarcted area was adjusted for edema using Reglodi's method: Edema adjusted (EA)-infarct volume: infarct volume×(contralateral hemisphere/ipsilateral hemisphere). Cumulative dead (white-stained) regions were combined from each brain to generate a total brain tissue infarcted volume score for each mouse.

Western blot analysis: In addition to preparing hPMSCs for injection, separate samples of these cells were tested for ACE2 expression. After the treatments described above, culture media were discarded, and hPMSCs were washed with ice-cold PBS and cells collected in Laemmli sample buffer (Bio-Rad; USA) with 10% 2-mercaptoethanol. The lysates were scraped using cell scraper and collected in microfuge tubes, and sonicated at 50% power for 15 sec, boiled at 95° C. for 15 min and stored at −80° C. 20 μl of protein was separated via SDS-PAGE, then immunoblotted to PVDF and incubated at 4° C. with rabbit anti-ACE2 (1:1000, Invitrogen; USA). Membranes were incubated with goat anti-rabbit IgG-HRP antibodies (1:2500, Sigma) for 2 h at 25° C. Signal was detected using ChemiDoc™ MP imaging system (Bio-Rad) and results analyzed with NIH Image-J software.

Statistical analysis: Statistical analysis was performed using GraphPad Prism software. For all experiments, data are expressed as mean±standard error of the mean (SEM). The statistical significance of the differences between groups was calculated using Student's t-test, one-way or two-way ANOVA with Bonferroni post-hoc tests where appropriate and indicated in the figure legend. A p-value<0.05 was considered statistically significant.

### Materials and Methods Part II

Study Design: The objectives of this study were to determine the mechanisms and extent to which hPMSCs protect the brain against acute ischemic injury in vivo, and to characterize barrier-stabilizing and anti-inflammatory effects of hPMSCs in vitro and in vivo. We used the Koizumi method of MCAO as an in vivo model of ischemic stroke in C57Bl/6 mice using a 1 h ischemic period following by 24 h reperfusion. hPMSC/EVs were injected (IP/IV) at the time of reperfusion to evaluate how hPMSC/EVs protect against IRI induced by MCAO. A sham group was used as control to evaluate how surgery and anesthesia contribute to observed results. CBF, infarct size, BBB integrity and neurological scores were measured in all experimental groups. We also used oxygen glucose deprivation/reperfusion (OGDR) conditions as our experimental in vitro model of ischemic stress where hPMSCs were contact-independently co-cultured with human brain endothelial cell to evaluate protective capabilities of hPMSCs on the in vitro barrier generated by human brain endothelial cell monolayers under normoxic and OGDR conditions. In general, we used n=5 to 10 mice per group for in vivo experiments and n=3 for in vitro experiments (with three replicates).

All histopathological analyses and evaluations (Nissl and Iba staining) were accomplished in a blinded fashion. Additionally, immunofluorescent imaging and analyses was performed using an automated evaluation approach (Image-J, Treatment groups (sham, MCAO groups as well as treatment groups) were performed on same days to help ensure equivalence and reliability. Assignment of hPMSC and/or EVs in these studies was selected based on availability of cells.

Surgery for MCAO model: Male mice (25-30 g) were anesthetized with ketamine (200 mg/kg i.p.)/xylazine (10 mg/kg i.p.). Once under deep anesthesia, middle cerebral artery occlusion (MCAO) was induced by creating a midline incision at the neck to expose the right carotid bifurcation. The right external carotid artery branch was isolated and ligated and the common carotid artery microclipped to permit creation of a small hole in the middle of the common carotid artery. A silicone-coated 6-0-nylon microfilament was introduced into the common carotid artery and the micro-clip released to allow advancement of the filament through the artery until the bulb-tip occluded the origin of the middle cerebral artery (MCA). This filament was left in place for 1 h (ischemia), and reperfusion initiated by withdrawal of the filament. For sham groups, vessels were cleared of overlaying connective tissue (also performed in MCAO) without further manipulation. The wounds were closed using surgical sutures (6-0) and mice allowed to recover from anesthesia. Postoperative monitoring of eating, drinking and movement were performed at 4 and 24 h following recovery.

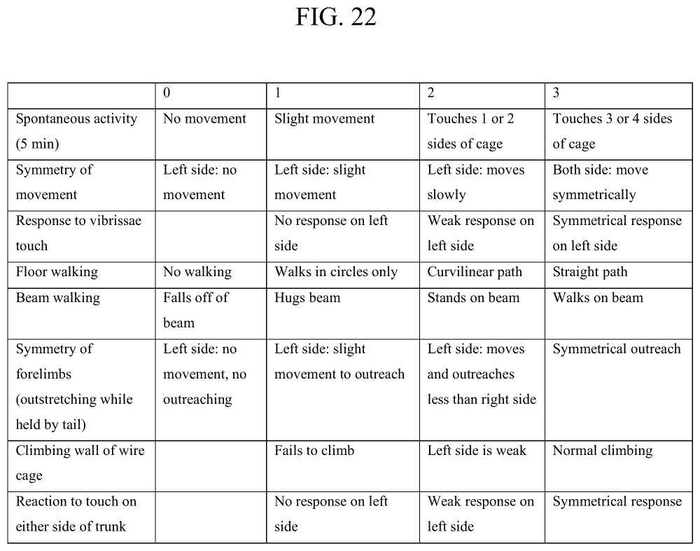

Neurological testing: Neurological outcomes were evaluated at 4 and 24 h after reperfusion using a 24-point scale (FIG. 22). Briefly, mice were given positive scores (0-3) for each of the following parameters: 5 mins of spontaneous activity, symmetry of movement and forelimbs (outstretching while tail is held), response to vibrissae contact, floor and beam walking, wire cage wall climbing, and reaction to touch on either side of the trunk.

hPMSCs isolation and culture: hPMSCs cells used in this study were isolated. Briefly, Placentas delivered by normal pregnant women were collected immediately after delivery. Since the placenta is considered medical waste, no consent from the patients was required. Villous tissue was separated by sterile dissection from different cotyledons, excluding chorionic and basal plates. After extensive washing with ice-cold phosphate-buffered saline (PBS), villous tissue was digested with trypsin (0.125% trypsin solution containing 0.1 mg/ml DNase I and 5 mM MgCl2) in Dulbecco's Modified Eagle's Medium (DMEM) at 37° C. for 90 min. Digested cells were collected and cultured in DMEM supplemented with 10% fetal bovine serum (FBS). PMSCs started to grow in 3-5 days. At ˜80% confluence, the cells were passaged with TrypLE™ Express (Invitrogen, Carlsbad, Calif., USA). hPMSCs were characterized using fluorescence-activated cell sorting (FACS) analysis or immunostaining. The primary antibodies used included mouse anti-human CD73 (BD Biosciences; USA), mouse anti-human CD90 (BD Biosciences; USA), mouse anti-human CD34 (BD Biosciences; USA), mouse anti-human HLA-DR (BD Biosciences; USA), mouse anti-human CD44 (Santa Cruz Biotechnology; USA), and mouse anti-human Oct-3/4 (Santa Cruz Biotechnology; USA). CD34-APC served as a negative control. hPMSCs were cultured in Dulbecco's-Modified Eagles's Medium (DMEM; Fisher Scientific; USA) with 10% (w/v) fetal bovine serum (FBS; Gibco; USA) and 1% penicillin/streptomycin (Sigma; USA) and used at passage 3-10. At confluency, hPMSCs cells were washed with PBS/EDTA, detached with 0.25% trypsin (Sigma; USA) for 2 mins, and subcultivated at a 1:3 split ratio.

IP Injection of hPMSCs: Trypsinized hPMSCs were washed twice with Ca⁺⁺/Mg⁺⁺ free HBSS and centrifuged (1500 RPM, 5 mins, 25° C.). 5×10⁵ hPMSCs were resuspended in 500 ml HBSS solution without Ca⁺⁺/Mg⁺⁺ and injected intraperitoneally (IP) into MCAO-treated mice at reperfusion.

One pre-clinical study suggested a dose of 5×10⁶ cells as the maximum number of cells that could be beneficial in rats, with higher doses causing high mortality reflecting emboli. Clinical trials consistently employ 10-20 million cells/kg of body weight. Since this was the inventors' first time evaluating protective effects of intraperitoneal injection of hPMSC in our study, we chose a dose near the higher end of the range used in humans and equivalent to that used in the rat study above as our starting reference. Therefore, we injected 5×10⁵ cells for 30 g BW (˜16.7 million cells/kg).

Inhibition and induction of EVs formation: To investigate effects of cholesterol depletion on hPMSCs-enhanced MCAO outcomes, 10 mM methyl beta-cyclodextrin (MβCD), was added to medium as a non-toxic cholesterol sequestering agent for 2 hours before harvesting the hPMSCs. Conversely, to enrich hPMSCs cholesterol/lipid content, culture medium was supplemented with synthetic cholesterol (1:250 ratio) (Gibco; USA) and CD lipid concentrate (1:100 ratio) (Gibco; USA) and incubated for 72 h at 37° C., 5% CO₂ prior to cell harvesting.

Trypan Blue exclusion test of cell viability: To determine the number of viable cells, hPMSC treated with or without MβCD were suspended in PBS containing 0.4% trypan blue in 1:1 ratio and incubated for ˜3 min at room temperature. 10 ml of trypan blue/cell mixture was applied to a hemocytometer. The unstained (viable) and stained (nonviable) cells were counted separately in the hemocytometer. To calculate the total number of viable cells per 1 ml of cell suspension, the total number of viable cells was multiplied by 2 (the dilution factor for trypan blue), then multiplied by 10⁴. The percentage of viable cells was calculated as follow: [total number of viable cells per ml/total number of cells per ml (viable+nonviable)]×100.

MTT assay: To evaluate the toxicity of MβCD on hPMSCs, MTT assay was performed. Briefly, hPMSCs were washed with PBS after removal of cell culture media. The cells were incubated at 37° C. with MTT (4,5-dimethylthiazol-2-yl)-2,5-diphenyltetrazolium bromide at the final concentration of 0.5 mg/ml cell culture media for 3 hours, when intracellular purple formazan crystals were visible under microscope. MTT was removed and absolute ethanol were added to the cells, followed by 30 min incubation at 37° C. until cells have lysed and purple crystal have dissolved. The absorbance was measured at 570 nm using Synergy H1 Hybrid Reader (BioTek; Vermont, USA). The absorbance reading of the blank was subtracted from all samples, and % viable cells was calculated as follow: [(Abs_{MbCD-hPMSC}−Abs_{blank})/(Abs_{hPMSC}−Abs_{blank})]×100.

Extracellular vesicle isolation: EVs were isolated. Briefly, culture media were collected from confluent hPMSCs 48 h after applying fresh medium. Unattached cells and debris were initially removed by centrifugation at 400×g for 10 mins (4° C.) and supernatants re-centrifuged at 20,800×g for 90 mins at 4° C. to pellet EVs. EVs pellets washed twice by centrifugation using 4° C. PBS/1 mM phenylmethylsulfonyl fluoride (PMSF) and pelleted at 20,800 g for 15 mins (4° C.). EVs pellets injected intravenously (2×10⁶ in 100 ml HBSS, Sigma; USA) into mice or evaluated by flow cytometry analysis.

Flow cytometry analysis: To evaluate hPMSC-released EVs by flow cytometry, freshly isolated EVs were resuspended in 100 ml Annexin-V Binding Buffer (BD Biosciences, San Jose, Calif.) and incubated with 5 mul of Annexin-V-FITC (BD Biosciences; USA) for 1 h at 4° C. under low-light conditions. 900 ml of 1× “Binding Buffer” was added to each sample. These samples were immediately collected on a 4 laser ACEA NovoCyte Quanteon Flow cytometer and data analyzed using NovoExpress 1.2 software. EV flow cytometric analysis was calibrated using Megamix-Plus FSC and Megamix-Plus SSC beads.

Fluorescence activated cell sorting (FACS) of EVs: To study effects of PS negative-EV protection in MCAO, the inventors isolated EVs from cholesterol-treated hPMSCs, separated PS negative-EVs by (FACS) based on fluorescent labeling used in MCAO therapy studies.

Cell localization using CytoID tracker: To track hPMSC in vivo, hPMSCs were first labelled using CytoID red long-term cell tracer kit (Enzo Life Science; USA). Briefly, cells were trypsinized and labelled with 1 ml of 2× CytoID for 5 min. Staining was stopped by adding 2 ml of stop buffer. Cells were centrifuged (400 g, 5 mins), cell pellets resuspended in 10 ml of complete media (DMEM+10% FBS+1% P/S) in a T75 flask and incubated at 37° C. for at least 12 h. CytoID-labelled hPMSCs were prepared and injected as described.

Laser Speckle measurement of blood flow: A Perimed Laser Speckle Imager (Pericam PSI HR; Sweden) was used to measure cerebral blood perfusion within the brains of different experimental groups. 24 h after reperfusion, mice under deep anesthesia were placed on a warm pad and the coronal skin removed and perfusion recordings accomplished using a high-resolution Laser Speckle camera (Perimed Laser Speckle Imager) at a working distance of 10 cm. ‘Perfusion’ reflects total cerebral flow signal measured in selected tissue regions of interest. Measurements are expressed as perfusion units (PU), using a fixed scale (arbitrary units).

TTC staining of infarcted tissue: 24 h after reperfusion, mice were deeply anesthetized with isoflurane and decapitated. The extent and severity of MCAO was evaluated after removal of the brain and staining of brain slices with 2,3,5-Triphenyltetrazolium chloride (TTC; Sigma; USA) to measure tissue viability and infarct size. After dissection, the brain was immersed in cold PBS for 10 m and sliced into 2.0 mm-thick sections using an anatomical slicer. Brain slices were incubated in 2% TTC/PBS for 30 m at 37° C. Areas of contralateral, ipsilateral, and infarction in each brain slice were recorded (Nikon 990) and measured using Image-j program (NIH). The infarcted area adjusted to the edema using Reglodi's method: (EA)-infarct volume: infarct volume×(contralateral hemisphere/ipsilateral hemisphere). Cumulative dead (white-stained) regions were combined from each brain to generate a total brain tissue infarcted score for each mouse.

Evans blue vascular permeability evaluation: BBB disruption following MCAO/reperfusion was measured by quantitating Evans blue (EB) transvascular leakage into the brain at 24 h. After MCAO, mice under deep anesthesia were injected with 100 ml of 2% EB, (4 mg/kg) through the femoral vein and allowed to circulate for 20 min before sacrifice. 0.2 ml blood was collected from the left ventricle and centrifuged at 5000 RPM for 10 mins to obtain plasma. Circulating dye was cleared by perfusing mice with 15 ml cold PBS. 10 ml plasma (supernatant) was added to 990 ml of 50% trichloroacetic acid (TCA; Sigma; USA), homogenized, sonicated and centrifuged (10,000 RPM) for 20 min. To extract EB from brain tissue, 2 ml of 50% TCA solution added to each brain, and the brain/TCA mixture homogenized and sonicated (amplitude 30, 10 W), and centrifuged at 10,000 RPM for 20 mins and finally diluted 3-fold with 100% ethanol. The amounts of EB in both plasma and brain tissue were quantified at 620 nm excitation and 680 nm emission using Synergy H1 Hybrid Reader (BioTek; Vermont, USA). EB leakage into brain tissue was normalized to the amount of EB in plasma.

Tissue preparation: 24 h after reperfusion, mice under deep anesthesia were cleared of blood with 15-20 ml of PBS. Brains were removed and post-fixed overnight in buffered 4% paraformaldehyde at 4° C. Brains were sectioned (30 μm sagittal slices) and mounted on slides.

Immunohistochemistry staining: Following deparaffinization, rehydration and antigen-retrieval with citrate buffer, 30 μm sagittal slices of brain tissue were incubated with 3% H₂O₂ (blocks endogenous peroxidase) and blocked with 1% bovine serum albumin (BSA; Sigma) and 4% normal goat serum in PBS-Triton (0.1%) for 1 h at 25° C. The sections were incubated with rabbit anti-Iba-1 antibody (1:1000, Wako Pure Chemical Industries; USA) at 4° C. overnight and treated with 2°-biotinylated anti-rabbit IgG (1:200 in 1% BSA/PBST; Vector Laboratories; USA) for 2 h at 25° C. The slices were incubated with Avidin Biotin Complex (R.T.U) (LifeSpan BioSciences; USA) reagent for 1 h at 25° C. followed by peroxidase substrate (Vector Laboratories; USA). Peroxidase activity was visualized with 3-diaminobenzidine. Slides were dehydrated with graded alcohols, cleared with xylene, and cover slipped.

Nissl staining: Tissue was fixed in 4% paraformaldehyde at 25° C. for 24 h. Sagittal brain sections (30 μm) were mounted on slides and Nissl staining performed. Samples were deparaffinized and rehydrated in decreasing ethanol concentrations. Slides were then processed for Nissl staining with thionin for ˜5 min at 25° C. Slides were dehydrated with graded alcohols, cleared with xylene and coverslipped. Nissl-stained images were recorded at 20× and 40×.

Immunofluorescence (IF) staining of brain tissues: CD31 and Human nuclear marker (Hu-Nu) expression were assessed using fluorescent staining. Paraffinized brain sections were rehydrated and blocked with 1% BSA and 5% goat serum in PBS for 1 h at 25° C. and incubated with rabbit anti-CD31 (1:100; Abcam), mouse anti-human nuclear antibody (1:100; Millipore) 12 h at 4° C. Following 4 washes in PBS (10 mins), sections were stained with AlexaFluor-488 goat anti-mouse (Life Technologies; USA), AlexaFluor-647 goat anti-rabbit (Life Technologies; USA) for 2 h at 25° C. Samples were washed 4× (10 mins) and mounted using DAPI/fluoroshield (Sigma; USA). Images were recorded (Nikon Eclipse E600FN, Tokyo, Japan); and processed with ImageJ software.

hCMEC-D3 isolation and culture: The hCMEC/D3 cell line (received from Dr. P. O. Couraud, INSERM) was isolated from temporal lobe microvessels of human tissue which was excised during surgery for control of epilepsy. The primary isolate enriched in cerebral endothelial cells (CECs) were sequentially immortalized by lentiviral vector transduction with the catalytic subunit of human telomerase (hTERT) and SV40 large T antigen. CEC were then selectively isolated by limited dilution cloning, and clones were extensively characterized for brain endothelial phenotype using endothelial markers including CD34, CD31, CD40, CD105, CD144 and von Willebrand factors (28). hCMEC-D3 cells were then cultured on collagen-coated plates using endothelial cell medium (EndoGRO; Millipore; USA) supplemented with MV complete culture media kit (Millipore; USA). When hCMEC-D3 cells reached 90% confluency, cells harvested using 0.25% trypsin (Sigma; USA), and centrifuged (1500RPM, 5 mins, 25° C.). Cells were counted and plated at appropriate densities for each experiment.

Immortalized human Placenta Mesenchymal Stem Cells (IhPMSC/IPSC): The inventors, recognizing the desire for large quantities of hPMSC for therapeutic IP administration and EV harvesting, investigated if immortalization of the hPMSC would offer a solution to limited human placentas, or if the immortalization process would be detrimental to the protective efficacy of the hPMSC. The inventors immortalized by lentiviral vector transduction with the SV40 large T antigen, and then tested these immortalized placental stem cells (IPSC) for efficacy in the MCAO model.

Oxygen Glucose Deprivation Reperfusion (OGDR): 1×10\hat{ }5 hCMEC-D3 cells were plated and grown to 80% confluency. After changing the media to glucose free DMEM+10% (w/v) FBS+1% P/S, the cells were incubated in a hypoxic chamber (1% O2) for 6, 12 or 16 h followed by 24 h reoxygenation in normal complete DMEM+10% FBS+1% P/S (5% O2).

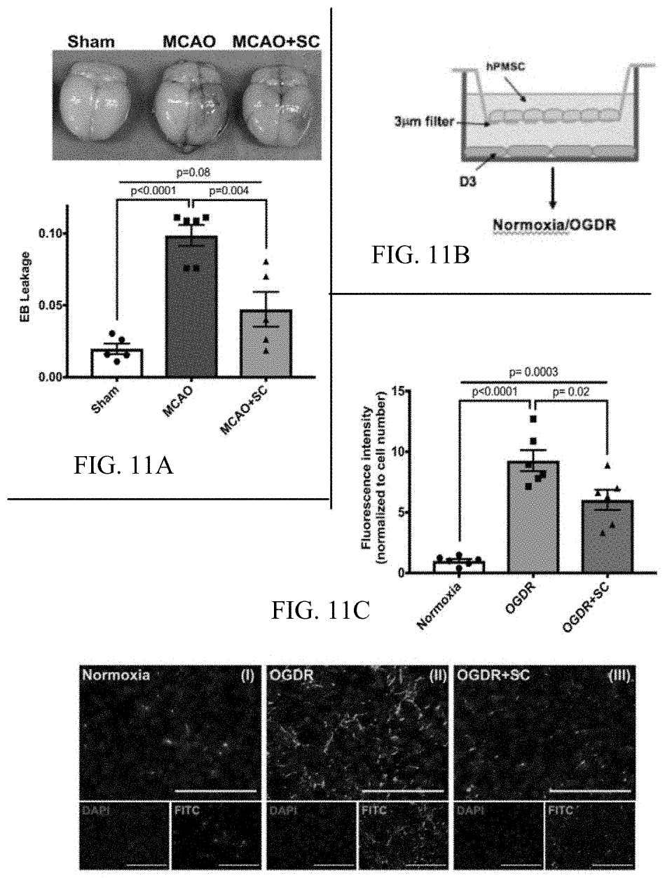

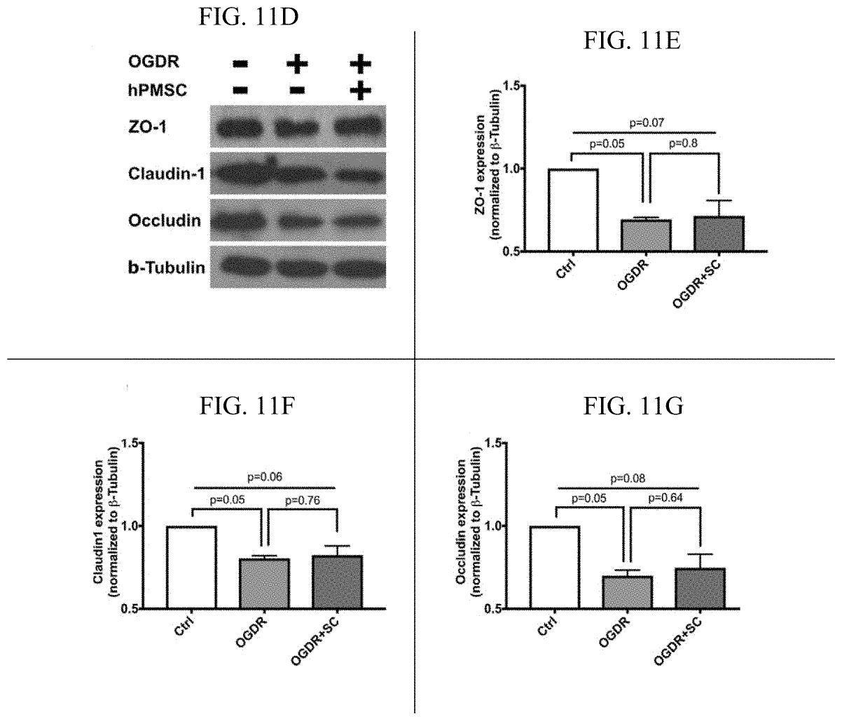

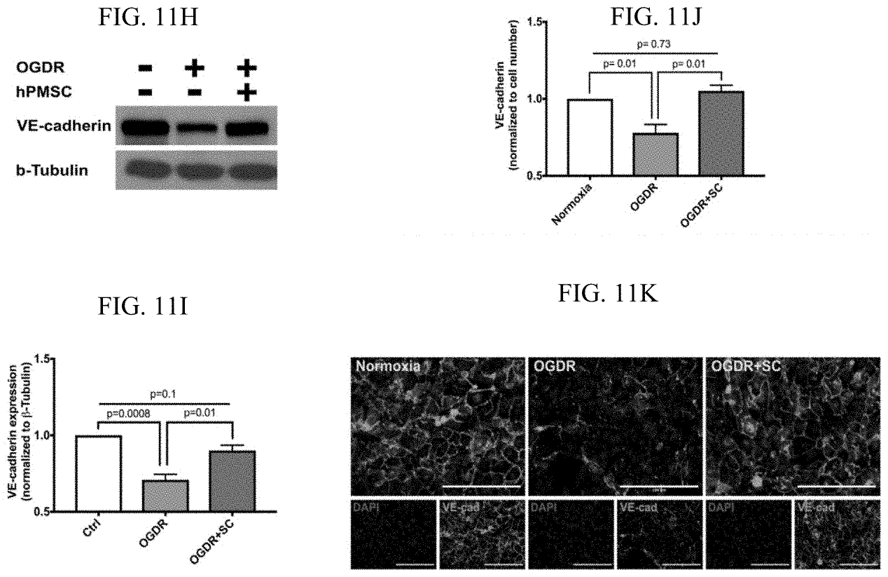

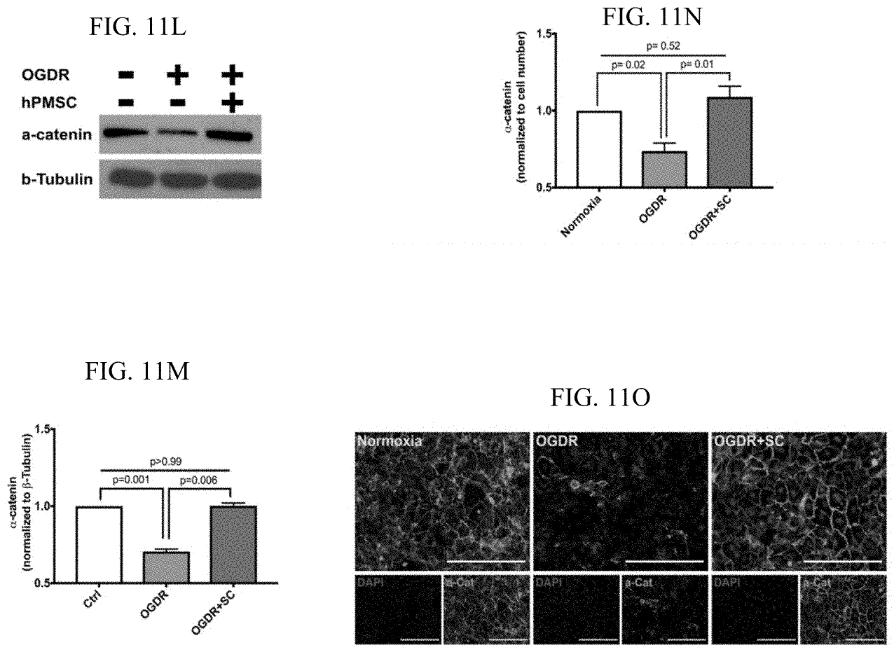

Transwell Co-culture Model: In this model (FIG. 11B), hCMEC-D3 cells were plated on the bottom chamber of transwell plates (Corning, USA) in 2 ml of complete media and hPMSCs cultured on the upper surface of cell culture inserts with permeable membrane (3 mm pore size). In this contact-independent model, hPMSCs cannot migrate between compartments and do not directly interact with hCMEC-D3 cells.

Biotinylated gelatin/FITC avidin permeability assay: To measure endothelial barrier function following OGDR, the inventors used the biotinylated gelatin/FITC avidin method as described in Ez-link-biotin protocol (Thermo Fischer; USA). Briefly, biotinylated gelatin solution added to 12-well plates and incubated at 4° C. overnight. After removing the biotinylated gelatin solution, hCMEC-D3 were plated at 2×10⁵ cells/well. FITC-avidin (1:50), (Life Technologies-Molecular Probes; USA) was added directly to the media and incubated for 3 min at 37° C. under low-light conditions. The cells were washed with 37° C. PBS twice, and fixed with 4% paraformaldehyde for 10 min at 25° C. Images were acquired using Nikon video imaging system Eclipse E600FN (Nikon, Tokyo, Japan) at 20× and processed with NIH-ImageJ software.

Western blot analysis: After desired treatments, cells collected in Laemmli buffer (Bio-Rad; USA) containing 10% 2-mercaptoethanol. The cells were scraped and sonicated at power of 50% for 15 sec, boiled at 95° C. for 15 min. 20 μl of protein was separated via SDS-PAGE, then immunoblotted to PVDF and incubated at 4° C. with rabbit anti-ZO-1 (1:500), rabbit anti-α-claudin-1 (1:500), (Invitrogen), rabbit anti-occludin (1:1000), rabbit anti-α-catenin (1:1000,Abcam), rabbit anti-VE-cadherin (1:1000) and rabbit anti-β-tubulin (1:2000,Cell Signaling). Membranes were incubated with goat anti-rabbit IgG-HRP antibodies (1:2500, Sigma) for 2 h at 25° C. Signal was detected using ChemiDoc™ MP imaging system (Bio-Rad) and results analyzed with NIH Image-J software.

IF staining of hCMEC-D3: For IF staining, hCMEC-D3 grown under normoxia/OGDR conditions were co-cultured with and without hPMSCs. The cells were washed with wash buffer (PBS+MgCl₂+CaCl₂+protease inhibitor), and then fixed in ice-cold 4% paraformaldehyde for 10 mins on ice, and permeabilized (0.5% Triton X-100/PBS, 5 mins, 25° C.). Cells were blocked with 5% BSA/5% goat serum for 1 h at 25° C. Primary antibodies (rabbit anti-α-catenin (1:100, Abcam) and rabbit anti-VE-cadherin (1:100, Cell Signaling) were diluted in wash buffer and incubated with cells overnight at 4° C. Cells were next incubated with fluorescently conjugated secondary antibody (AlexaFluor 488 goat anti-rabbit; Life Technologies; USA) for 1 h and rinsed twice. Hoechst (Thermo Scientific; USA) was added to the cells for 5 mins, washed, mounted on glass slides and images recorded using a Nikon video at 20× magnification. Images were processed with Image-J software.

Statistical analysis: Statistical analysis was performed using GraphPad Prism software. For all experiments, data are expressed as mean±standard error of the mean (SEM). The statistical significance of the differences between groups was calculated using Student's t-test, one-way ANOVA with Bonferroni post-hoc test or two-way ANOVA with Sidak's multiple comparisons tests where appropriate and indicated in the figure legend. Flow cytometry data were analyzed using Mann Whitney U test. A p-value<0.05 was considered statistically significant.

Ethics Statement: Mice: All animal protocols were approved by the LSUHSC-S Institutional Animal Care and Use Committee (IACUC) according to NIH guidelines. We used male C57BL/6J mice (Jackson Laboratories, Bar Harbor, Me.) in all studies at 9-16 weeks of age. Animals were housed in a barrier facility and maintained on a normal diet. Human Placenta: Collection of human placentas for MSC isolation was approved by the IRB at Louisiana State University Health Science Center-Shreveport (LSUHSC-S), and MSC isolation was processed at the Department of Gynecology and Obstetrics, LSUHSC-S. Human brain endothelial cells (hCMEC/D3): The hCMEC/D3 cell line was provided by Dr. P. O. Couraud. Cells were isolated from section of brain tissue removed during surgery for epilepsy following informed consent according to protocols established at INSERM, Institut Cochin, France.

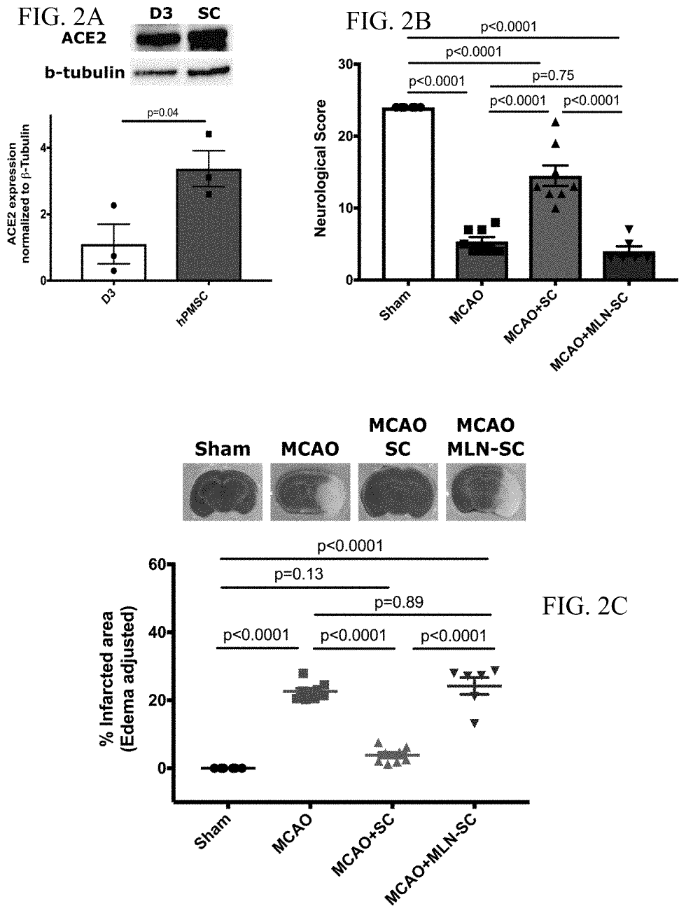

RESULTS: Inhibition of ACE2 prevents the protective effects of hPMSC in the MCAO model: ACE2 was hypothesized to provide some stroke protection, reducing infarct size and improving neurological function in endothelin-1-induced stroke models. The inventors found that hPMSCs express greater than 3 times more ACE2 (3.37±0.54) than human brain endothelial cells (hBMEC-D3) (1.10±0.59, p=0.04; FIG. 2A).

The inventors previously demonstrated that intraperitoneal (IP) administration of hPMSCs at the time of reperfusion in the MCAO model of ischemic stroke produced highly significant preservation of the ipsilateral hemisphere characterized by almost complete inhibition of cerebral infarction, significant preservation of CBF within the post-MCAO brain, and improvement of neurological function. To evaluate contributions of hPMSCs-derived ACE2 in brain protection after ischemic stroke, the inventors inhibited ACE2 activity in hPMSCs using 10 μM MLN-4760, a highly potent and selective ACE2 inhibitor. While IP injected hPMSC (non-treated 5×10⁵ cells in 500 ml HBSS) into the MCAO mice at the time of reperfusion improved the neurological function of MCAO mice (14.5±1.42, p<0.0001), MLN-treated hPMSCs (5×10⁵ cells in 500 ml HBSS) (1 hour following ischemia) did not provide significant protection against neurological deficits (4±0.68), (FIG. 2B). Infarcted areas in MLN-hPMSCs treated MCAO mice were more than 6 times larger (24.19±2.48) than in mice treated with hPMSCs-treated MCAO group (3.84±0.80, p<0.0001) (FIG. 2C). The infarct size of the MCAO mice group injected with MLN-treated hPMSC (ACE2 inhibited) were similar to that of MCAO mice not receiving hPMSC therapy (22.62±2.03, p=0.89).

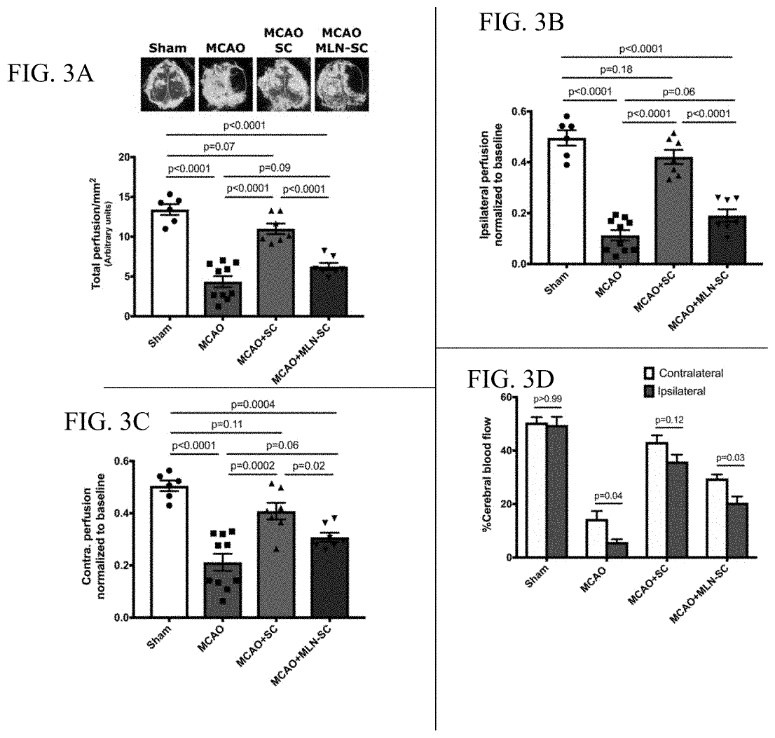

To determine how MLN-treated hPMSC affect cerebral blood flow following MCAO induced strokes, we measured cerebral perfusion using Laser Speckle Contrast Imaging. The inventors found that total cerebral blood flow was significantly reduced in MLN-hPMSCs treated MCAO mice (6.25±0.45, p<0.0001) compared to hPMSC-treated MCAO (10.99±0.66) and sham groups (13.42±1.01, p<0.0001; FIG. 3A).

Normalizing ipsilateral or contralateral blood flow to baseline levels (values obtained from averaged sham total CBF), we found significant decreases in both ipsilateral (0.19±0.30 vs. 0.49±0.07 in sham group, p<0.0001; FIG. 3B) and contralateral (0.31±0.19 vs 0.5±0.09 in sham group, p=0.0004; FIG. 3C) hemispheres of MLN-hPMSC treated MCAO mice which was comparable to untreated MCAO group (ipsilateral: 0.11±0.03, p=0.06 (FIG. 3B); contralateral:0.21±0.04, p=0.06 (FIG. 3C)).

As is shown in FIG. 3D, brain blood perfusion in both hemispheres of the sham (50% in contralateral vs 49.6% in ipsilateral; p>0.99) and hPMSC-treated MCAO (43% in contralateral vs 36% in ipsilateral; p=0.13) groups were comparable (FIG. 3D). This normal perfusion significantly shifted towards the contralateral hemispheres in both MCAO (14.5% in contralateral vs. 6% in ipsilateral; p=0.04) and MLN-hPMSC treated MCAO (30% in contralateral vs. 20% in ipsilateral; p=0.03) groups (FIG. 3D), suggesting that hPMSC lose their ability to maintain brain blood flow post MCAO after MLN treatment to inhibit ACE2 activity in hPMSC.

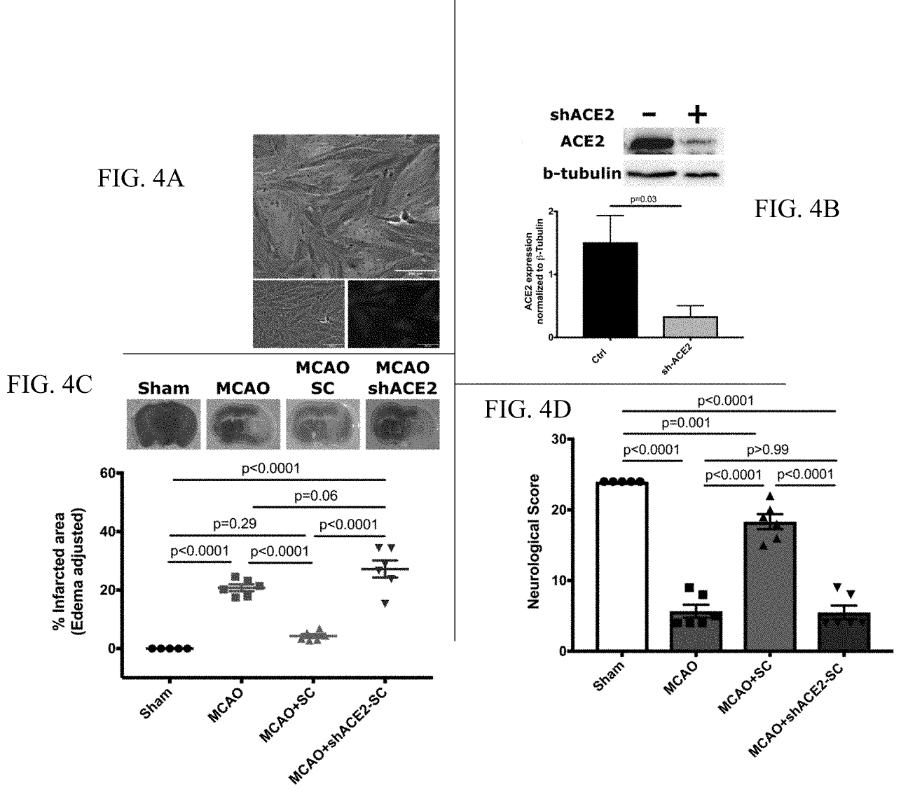

To further validate the inventors' findings for a role for hPMSC-derived ACE2 in protection against stroke injury, hPMSCs were transfected with lentivirus-shACE2-EGFP which reduces expression of ACE2 in hPMSCs. Lentiviral transfection efficiency was confirmed by both fluorescent microscopy (FIG. 3A) and by western blot analysis (FIG. 4B). Similar to the inventors' pharmacological inhibition of ACE2 expression by MLN-4760 (FIGS. 2 & 3), IP injection of shACE2-hPMSCs (5×10⁵ cells in 500 ml HBSS) failed to provide the significant protection against tissue injury (TTC staining; 27.18±2.93) or neurological impairment (5.5±0.96) that was observed in hPMSCs-treated MCAO mice (infarction: 20.66±3.23, p<0.0001 (FIG. 4C); neurological score: 18.33±1.05, p<0.0001 (FIG. 4D).

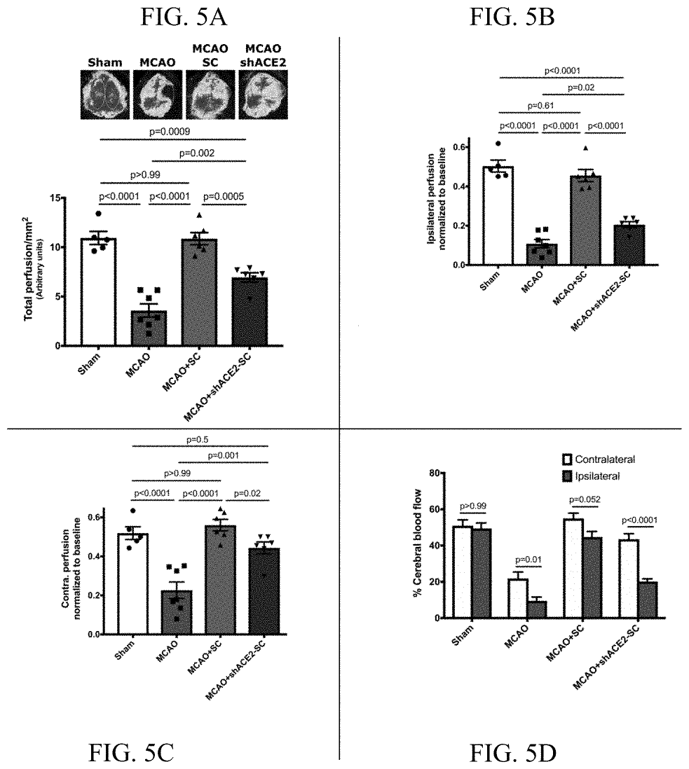

Furthermore, injection of shACE2-hPMSCs did not correct blood flow disturbances induced by MCAO (FIGS. 5A-C). As seen with Laser Speckle imaging, total cerebral perfusion was reduced to 6.94±0.47 in shACE2-hPMSCs treated MCAO group compared to 10.87±0.61 in hPMSCs treated group (p=0.0005; FIG. 5A). Normalizing this value to baseline level, the inventors found a significant reduction in brain blood flow in both ipsilateral (0.21±0.0 vs 0.46±0.03, p<0.0001; FIG. 5B) and contralateral (0.44±0.03 vs 0.56±0.03, p=0.02; FIG. 5C) hemispheres of shACE2-hPMSCs treated MCAO compared to MCAO mice treated with control hPMSC. shACE2-hPMSCs also failed to protect against CBF imbalances observed between ipsilateral (20% perfusion) and contralateral (44%, p<0.0001; FIG. 5D) hemispheres compared to control hPMSCs given to mice with MCAO (45% in ipsilateral and 54% in contralateral, p=0.052).

In summary, the inventors' data are consistent with stem cell derived ACE2 playing a significant role in mediating the maintenance of brain perfusion and protection following intraperitoneal hPMSC administration after stroke.

hPMSC-derived ACE2 protects the brain against ischemic injury independent of the AT2R pathway: While the inventors' data supported a significant role for hPMSC-derived ACE2 activity in the protection afforded by hPMSCs against MCAO (FIGS. 2-5), it was still unclear which ACE2 derived factors and receptor(s) (AT2R or masR) actually contributed to ACE2-mediated protection offered by hPMSCs.

To investigate the relative contributions of ACE2-Ang 1-7 axis receptors (AT2R vs. MasR) involved in the protection mediated by hPMSC in the MCAO stroke model, first, the inventors blocked AT2R by administering PD123319 (10 mg/kg intravenously) to mice 1 hour before MCAO surgery.

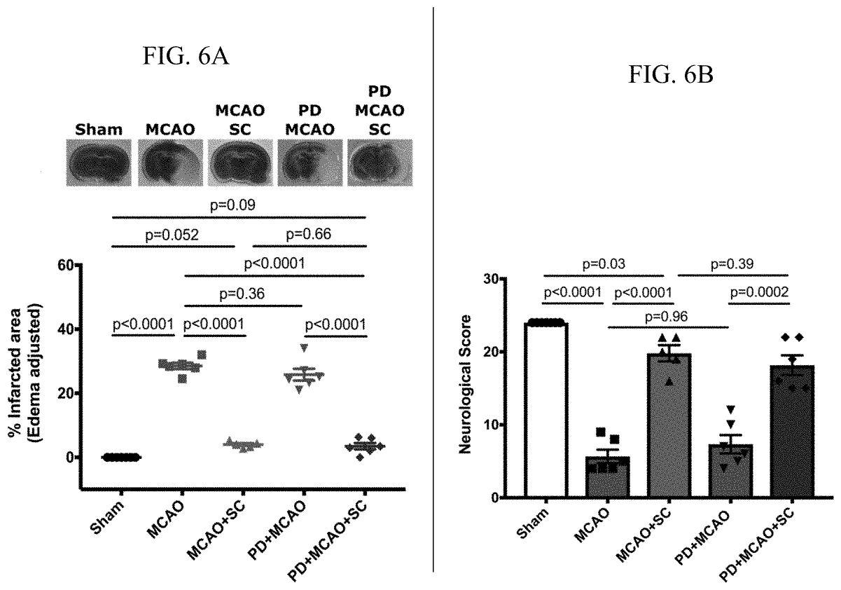

TTC staining, neurological scores and laser speckle imaging of these experimental groups showed that AT2R does not appear to contribute to any beneficial effects of hPMSC-derived ACE2. As illustrated in FIG. 6A, infarcted areas in PD-pretreated MCAO mice (25.8%) were comparable to untreated MCAO group (28.5%, p=0.36). Furthermore, hPMSCs administered to PD-pretreated MCAO mice were still able to provide significant protection against ischemic stroke injury (MCAO), as shown by the significant reduction in infarction size (reduced to 3.47±0.98, p<0.0001). Neurological function in PD-pretreated MCAO mice (7.3±1.25) was also significantly improved to 18.17±1.35 in hPMSC treated PD-MCAO mice (p=0.0002; FIG. 6B).

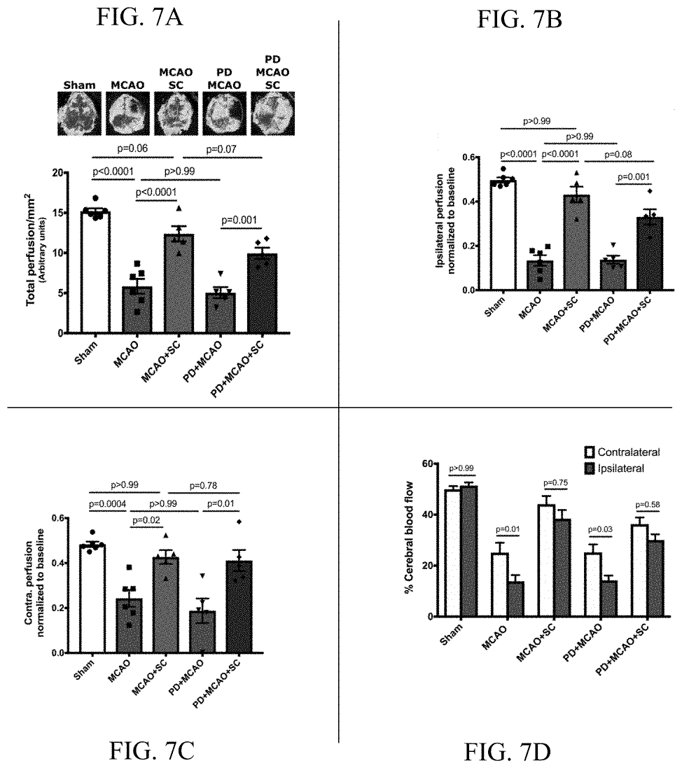

Additionally, hPMSCs produced recovery of cerebral blood flow, after PD-pretreatment in MCAO mice (9.95±0.96), to the level observed in hPMSC-treated MCAO group (12.38±0.70, p=0.08). This was significantly higher than brain perfusion observed in the PD-pretreated MCAO group (5.04±0.69, p=0.001; FIG. 7A). The decreased levels of blood flow seen in the ipsilateral (0.14±0.02) and contralateral (0.19±0.05) hemispheres of PD-pretreated MCAO mice were not seen after hPMSC administration (ipsilateral: 0.33±0.03, p=0.001; contralateral: 0.41±0.05, p=0.01) (FIGS. 7B-C). Administration of hPMSC further normalized blood distribution between the ipsilateral (30%) and contralateral (36%) hemispheres of PD-pretreated MAO group (p=0.58) compared to untreated PD-MCAO mice (ipsilateral: 14%, contralateral: 25%; p=0.03) (FIG. 7D).

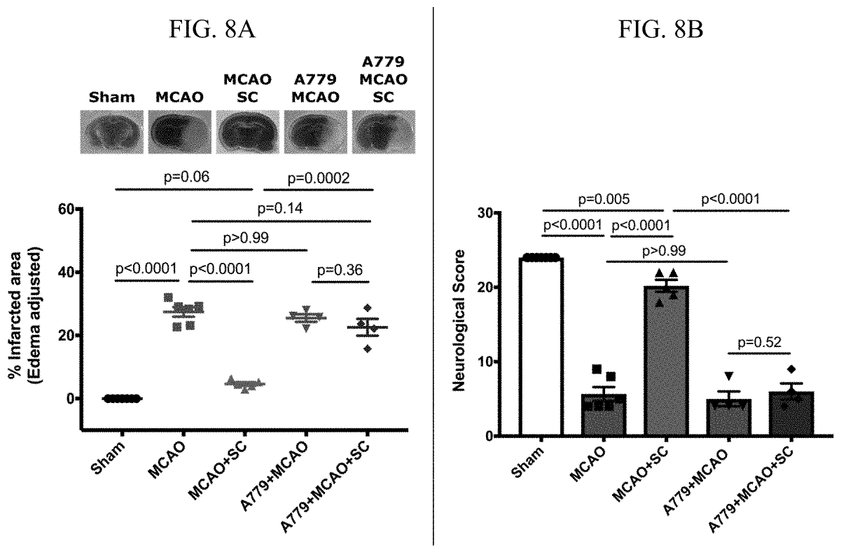

hPMSCs-ACE2 based protection is mediated by the masR pathway in the MCAO stroke model: To test how the masR pathway might contribute to the protection provided by ACE2, the inventors pre-treated mice with masR antagonist A779 (80 mg/kg i.p) 1 hour before MCAO surgery. Interestingly, TTC staining revealed no significant protection against infarct development in A779-pretreated MCAO mice (22.58±2.67) after IP injection of hPMSCs (25.49±1.22, p=0.36; FIG. 8A). Neurological scores were similar in both A779-pretreated MCAO (5±1) and hPMSC injected A779-pretreated MCAO groups (6±1.08, p=0.52; FIG. 8B).

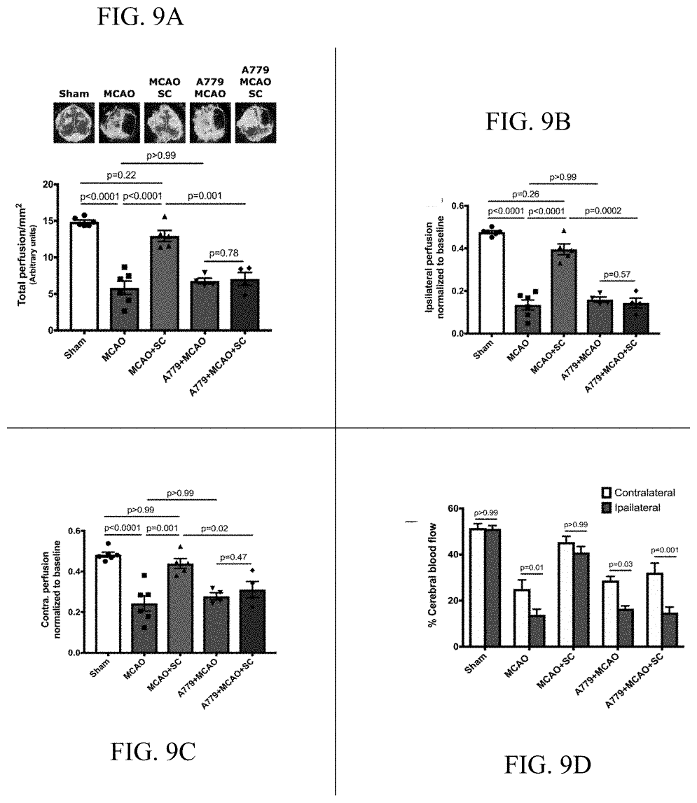

Total cerebral blood flow (7.05±0.88, p=0.78), ipsilateral perfusion (0.16±0.01, p=0.57), and contralateral perfusion (0.28±0.02, p=0.47) in A779-pretreated MCAO mice were found to be comparable to their respective hPMSCs treated groups (6.78±0.39, 0.14±0.02, and 0.31±0.04; FIG. 9A-C). In addition, hPMSCs injection failed to protect against the perfusion imbalances that occurred between ipsilateral and contralateral hemispheres of A779-pretreated MCAO group. As shown in FIG. 9D, relative perfusion to the contralateral (32%) remains significantly greater compared to ipsilateral (15%) (p=0.0008) hemispheres of A779-pretreated MCAO mice even after hPMSC administration.

Cumulatively, these data are consistent with hPMSCs-associated ACE2 mediating its protection via the masR pathway, since the masR antagonist (A779), but not AT2R antagonist (PD 123319), eliminated the beneficial effects of hPMSCs in protecting against tissue injury and blood flow dysregulation after stroke.

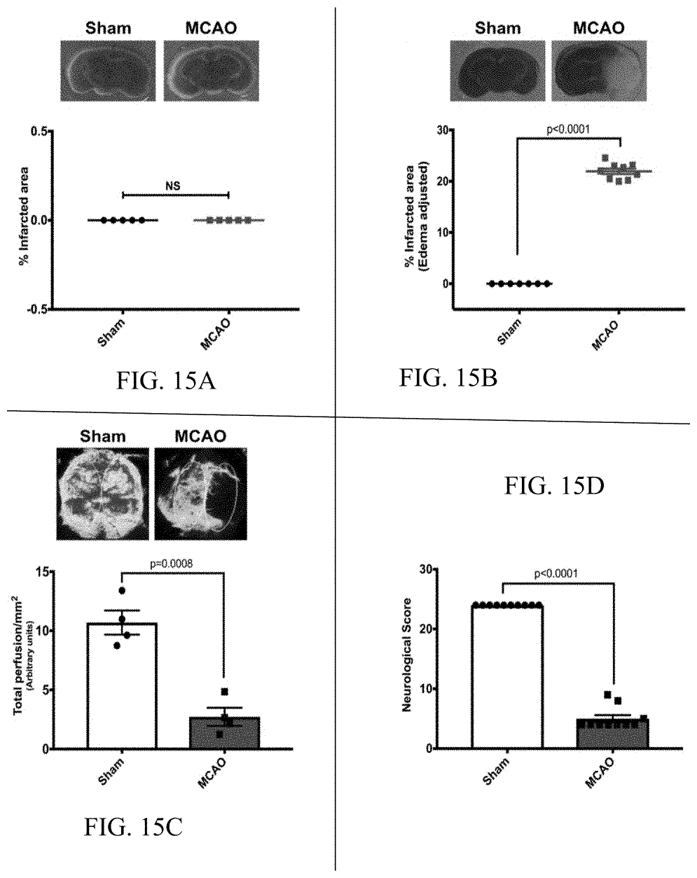

MCAO model of stroke: the inventors induced ipsilateral cerebral hemispheric ischemic injury in C57Bl/6 mice using MCAO model based upon prior studies of this model which were associated with an 88 to 100% rate of infarction. Duration of reperfusion is another key factor influencing pathophysiology and outcomes in the MCAO model. TTC staining was used to identify the areas of infarction. Initial studies revealed no consistent infarct pattern 4 hours after reperfusion (FIG. 15A) which led to the ongoing protocol of one hour of ischemic and then sacrifice of the animal with infarct assessment at 24 hours. The inventors observed, in preliminary studies, an infarct volume of 21.94±0.52% vs 0 infarct in the sham animals (p<0.0001; FIG. 15B). Reduced CBF following MCAO impairs motor function and survival. In agreement with this, the inventors also observed reduced CBF in our MCAO group at 10.69±1.017 for sham animals compared to 2.723±0.764 (p=0.0008; FIG. 15C) which was accompanied by substantially decreased neurological scores 5.0±0.59 in MCAO (impaired motor function) compared to 24.0±0 in sham mice (p<0.0001; FIG. 15D).

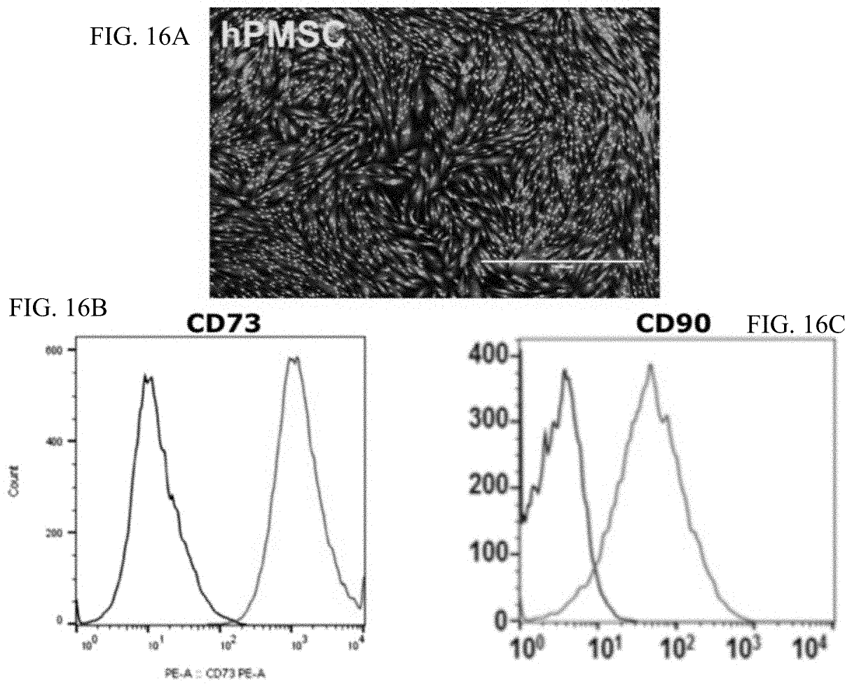

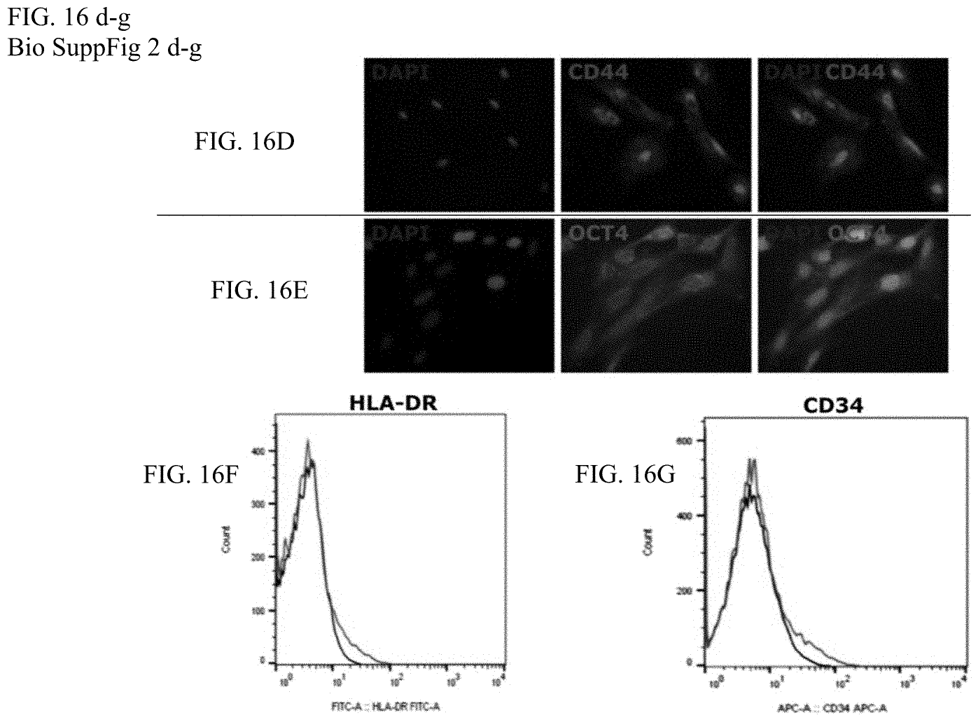

Characteristics of hPMSCs: In cell culture environment, hPMSCs are fibroblast-like, “spindle”-shaped, plastic-adherent cells that exhibit robust in vitro expansion (FIG. 16A). To validate the potential for hPMSCs to replicate stem cell neurorestorative capability, the inventors measured the expression of classical MSC markers CD73, CD90, CD44 and Oct4 by FACS analysis and immunostaining (FIG. 16B-E). hPMSCs did not express high levels of HLA-DR, or the hematopoietic lineage marker (CD34) (FIG. 16F-G), which is the major criteria to define MSCs.

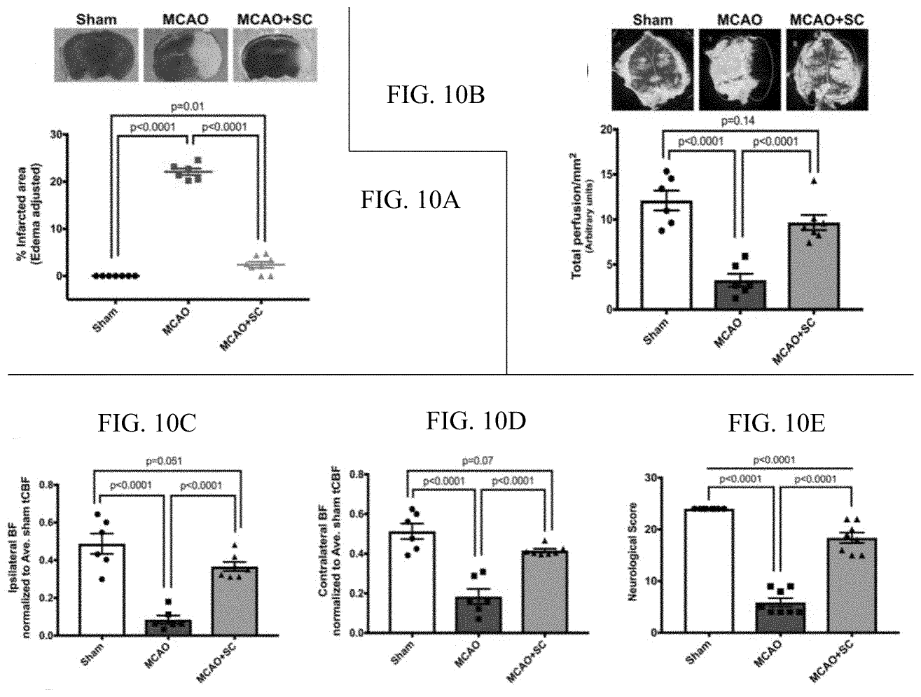

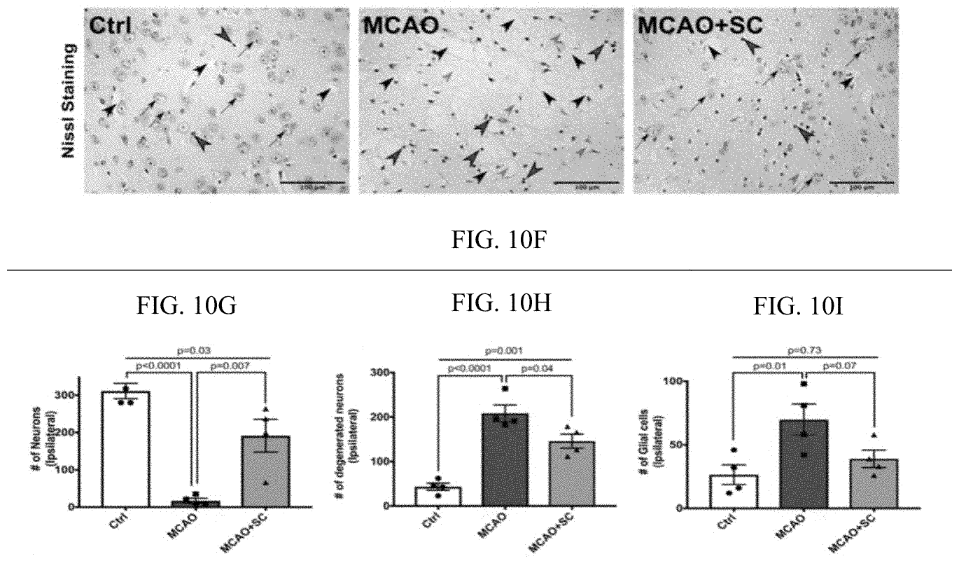

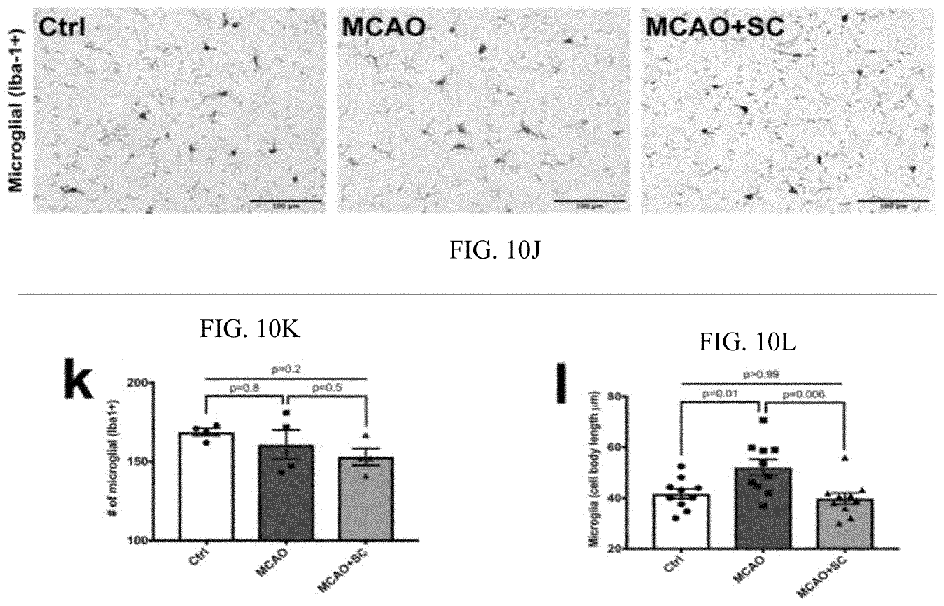

IP injection of hPMSCs preserves ipsilateral hemisphere viability and perfusion in the MCAO model: The inventors assessed the potential of IP injection of hPMSCs, upon cerebral reperfusion, to help to preserve cerebral tissue integrity following the ischemic insult. Strikingly, using TTC staining, the inventors observed that IP administration of 5×10⁵ hPMSCs significantly reduced ischemic injury in our model (2.37±0.74%; p<0.0001) towards levels observed in sham (0±0) compared to MCAO without SCT (22.08±0.79%) (FIG. 10A).

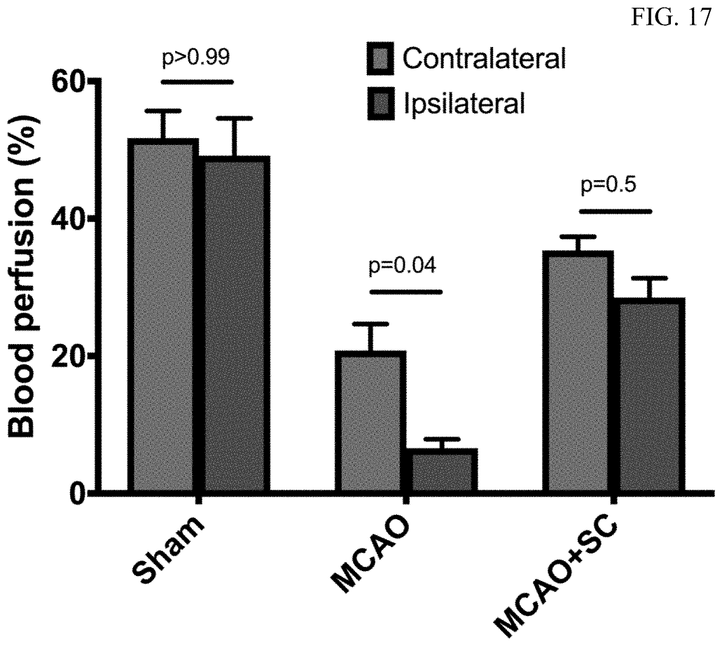

To determine the extent of hPMSCs ability to preserve CBF at 24 h after IP-SCT in the MCAO mice, the inventors measured cerebral perfusion using Laser Speckle Contrast Imaging. There was significant preservation of CBF in our hPMSC-treated group (9.657±0.85 vs 3.24±0.72 in untreated post-MCAO) (p=0.0001; FIG. 10B). Normalizing either ipsilateral or contralateral blood flow to the average sham total CBF value as the reference point, revealed significant decreases in both ipsilateral (0.08±0.02; p<0.0001; FIG. 10C) and contralateral (0.18±0.04; p<0.0001; FIG. 10D) hemispheres of untreated MCAO animals. Most interestingly, when comparing CBF between both hemispheres, there was a significant shift in the balance of blood flow towards the contralateral hemisphere of MCAO mice. As shown in FIG. 17, blood perfusion in both hemispheres of the sham group was comparable (51.71% in contralateral vs. 49.17% in ipsilateral; p>0.99). This balance significantly shifted towards the contralateral hemisphere in the MCAO group (20% in contralateral vs. 6% in ipsilateral; p=0.04). Remarkably, MCAO-induced blood flow disturbances were restored with hPMSC treatment (35% in contralateral vs. 29% in ipsilateral; p=0.5), indicating a redistribution of CBF associated with improved post-MCAO tissue survival in hPMSC-treated mice.

Consistent with abundant evidence showing that reduced CBF after MCAO chronically impairs neural function and survival, the inventors also observed that neurological function was significantly maintained in IP-hPMSC treated MCAO mice (18.38±1.01) compared to untreated MCAO group (5.87±0.83) (p<0.0001; FIG. 10E).

Destruction of neurons is another hallmark of ischemic stroke injury which may be improved by SCT. To discriminate viable from degenerating neurons and glia in the striatum of both ipsilateral and contralateral hemispheres, we performed modified Nissl staining (FIG. 10F-10I). Nissl staining revealed reduced numbers of viable neurons in the ipsilateral hemisphere compared to the contralateral side (16.75±7.12; p=0.02) of MCAO group. This degree of neuronal injury was attenuated by IP-hPMSC following MCAO (191.5±43.91) (black arrows; FIG. 10F, quantification; FIG. 10G). In comparing ischemic damaged neurons with enlarged intercellular spaces, reflected of cellular destruction in stroke, there was significant reduction in degree of insult with hPMSC-treated mice (146±15.83 vs 208.3±18.81; p=0.04) (black arrows head; FIG. 10F, quantification; FIG. 10H). Many unstained (‘spongiform’) regions were also observed in MCAO-treated brains; this appearance was not detected in any IP-hPMSC treated MCAO brains (red arrows; FIG. 10F). In addition, the proliferation of reactive glial cells (red arrows head; FIG. 10F), was reduced from 69.75±12.36 in the untreated ipsilateral hemispheres of MCAO mice compared to 39±6.86 with IP-hPMSC (p=0.02, quantification; FIG. 10I).

Early activation of microglia (resident immune cells in the CNS), is a key neuroimmunological responses to a wide variety of pathological stimuli e.g., trauma, inflammation, degeneration, ischemia. Ionized calcium binding adaptor protein (Iba-1), is specifically mobilized in microglia after inflammation and plays important roles in microglial regulation/activation. In recognition that inflammatory cell invasion is part of the pathogenesis of evolving cerebral ischemia, the inventors assessed microglia activation via immunohistological staining with anti-Iba-1 antibody (FIG. 10J-10L). Iba-1⁺ microglia were counted in the border areas between cortex and striatum of each hemisphere. The inventors found the number of microglia (Iba-1⁺) within the ipsilateral hemispheres with administration of hPMSCs following MCAO, was similar to that in the MCAO group, 153±5.33 and 160.8±9.31, respectively (p=0.5) (FIG. 10J and 10K). However, elongated microglial cell bodies observed in MCAO mice (52.02±3.23) was significantly (p=0.002) reduced by IP-SCT (39.84±2.23) (FIG. 10J and 10L). This supports hPMSCs administration inhibiting microglial activation with presumably a salutary effect on the ischemic insult.

hPMSCs maintain blood brain barrier integrity against MCAO in mice: To characterize changes in BBB integrity in MCAO-stressed brains, Evans blue (EB) vascular permeability analysis was performed. FIG. 11A shows that EB leakage significantly increased in the ipsilateral hemisphere of MCAO group (0.09±0.007 vs sham:0.02±0.01; p=0.0001) shown by blue tissue stain (FIG. 11A); indicating that BBB function was lost. In contrast, 24 h after IP administration of 5×10⁵ hPMSCs, BBB integrity was maintained as shown by low EB uptake (0.04±0.01; p<0.0001) into the ipsilateral hemisphere compared to MCAO group (FIG. 11A).

The inventors' previous experiments showed that oxygen glucose deprivation (OGDR) increases endothelial permeability; similar stresses in stroke may impair BBB function. The inventors therefore investigated whether hPMSCs could maintain the in vitro barrier formed by human brain endothelial cell against OGDR (schematic FIG. 11B). Using a biotinylated gelatin FITC-avidin permeability assay, the inventors found that OGDR significantly increased FITC-avidin permeability (9.26±0.87, p=0.0001; FIG. 11C), and that hPMSCs contact-independently stabilized hCMEC-D3 barrier integrity against OGDR stresses (6.03±0.83, p=0.02; FIG. 11C).

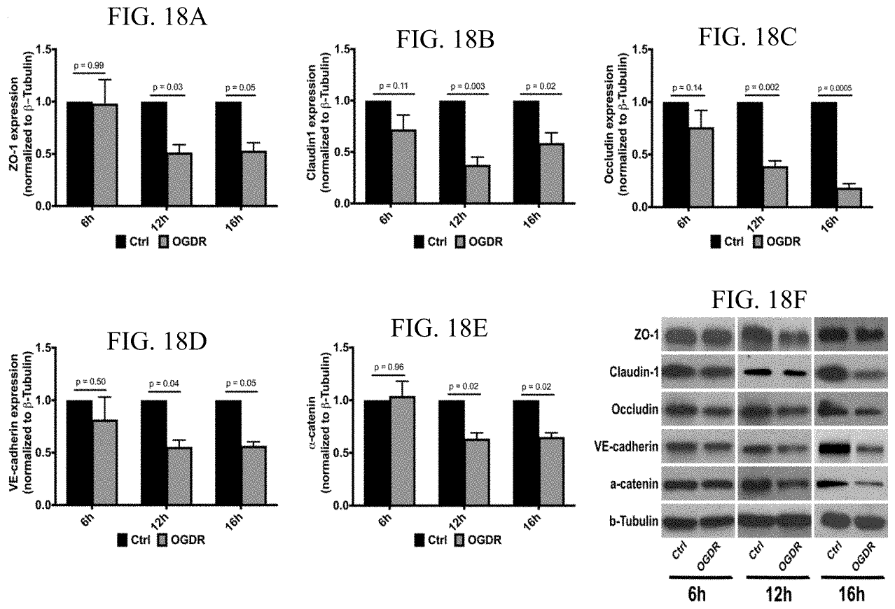

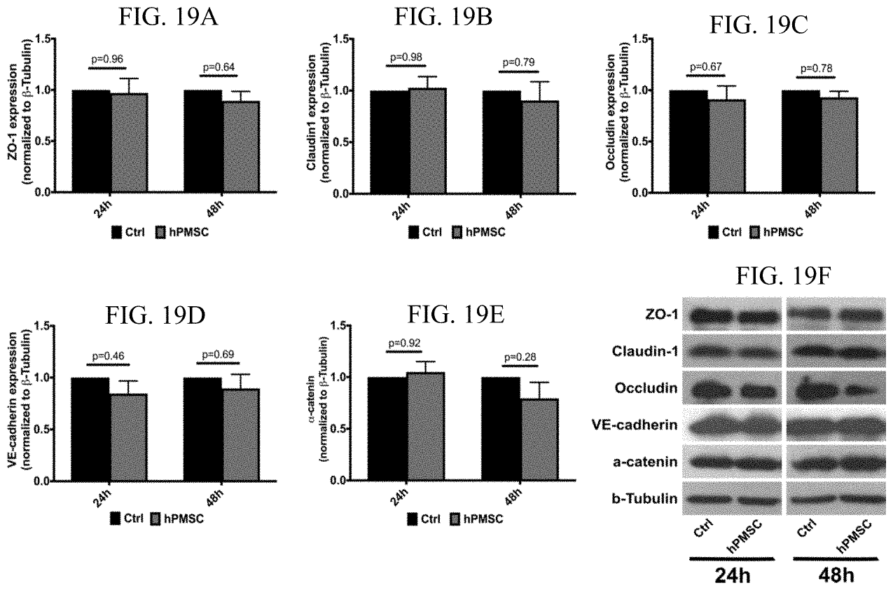

The inventors' previous studies showed that increased endothelial permeability following ischemia reflects alterations in organization of tight/adherens junctional (TJs/AJs) proteins (e.g. occludin, claudins, VE-cadherin, catenins). The inventors assessed the impact of OGDR on TJ/AJ protein expression at 6, 12 and 16 h. FIGS. 18A-18F reveal significant reductions in the abundance of ZO-1; (p=0.03), Claudin-1; (p=0.02), Occludin; (p=0.0005; FIGS. 18A-18C) and VE-cadherin; (p=0.05), α-catenin; (p=0.02; FIGS. 18D-18E) after 16 h incubation of hCMEC-D3 monolayers under OGDR stress. The inventors chose 16 h time point of OGDR for following in vitro experiments. The inventors next evaluated whether hPMSCs could prevent loss of TJs/AJs in hCMEC-D3 monolayers under both normoxia and OGDR. The expression level of tight junctional proteins e.g., ZO-1 (p=0.8; FIGS. 11D and 11E), claudin-1 (p=0.76; FIGS. 11D and 11F) and occludin (p=0.64; FIGS. 11D and 11G) did not change significantly under OGDR (FIGS. 11D-11G) nor normoxia (FIGS. 19A-19C, and 19F) with hPMSCs. However, the inventors found that hCMEC-D3 monolayers expressed significantly more VE-cadherin (p=0.01; FIGS. 11H and 11I) and α-catenin (p=0.006; FIGS. 11L and 11M) under OGDR conditions when contact-independently co-cultured with hPMSCs); this was not observed with stem cells under normoxic conditions (FIGS. 19D-19F). The spatial localization of VE-cadherin (FIG. 11K) and α-catenin (FIG. 11O) in D3 monolayers improved with hPMSC treatment under OGDR stress. Additionally, VE-cadherin (p=0.01; FIGS. 11J and 11K) and α-catenin (p=0.01; FIGS. 11N and 11O) abundance increased in hCMEC-D3 junctions under OGDR at 24 h after hPMSCs treatment. Taken together, the inventors' findings indicate that hPMSC factors improve brain endothelial barrier function by apparently enhancing endothelial junctions after ischemic stress.

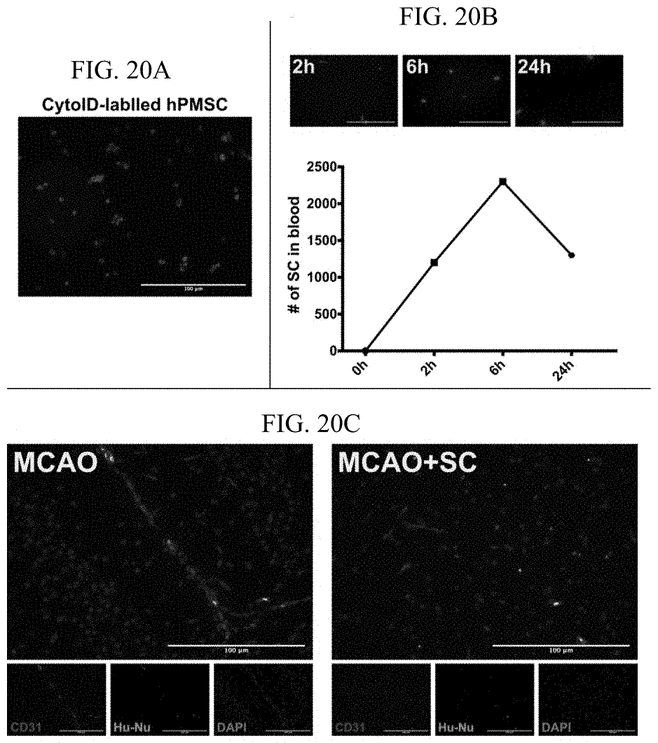

hPMSC-released EVs contribute to SC-based protection in the MCAO model: To determine numbers of hPMSCs in the bloodstream, CytoID-dye labeled hPMSCs (FIG. 20A) were IP-injected into the MCAO mice, and blood collected at 2, 6, and 24 h post-injection. Interestingly, the inventors found extremely low numbers of circulating hPMSCs (maximum of 2300 cells in 1 ml blood at 6 h; FIG. 20B). The inventors next examined brain tissue for the presence of hPMSCs using anti-human nuclear antibody to detect hPMSCs; mouse CD31 was used as a positive control. The inventors did not detect any hPMSCs penetrating the brain (FIG. 20C), consistent with beneficial effects of hPMSCs mediated by paracrine signaling pathways restricted to the vasculature rather than cell-integration into the brain.

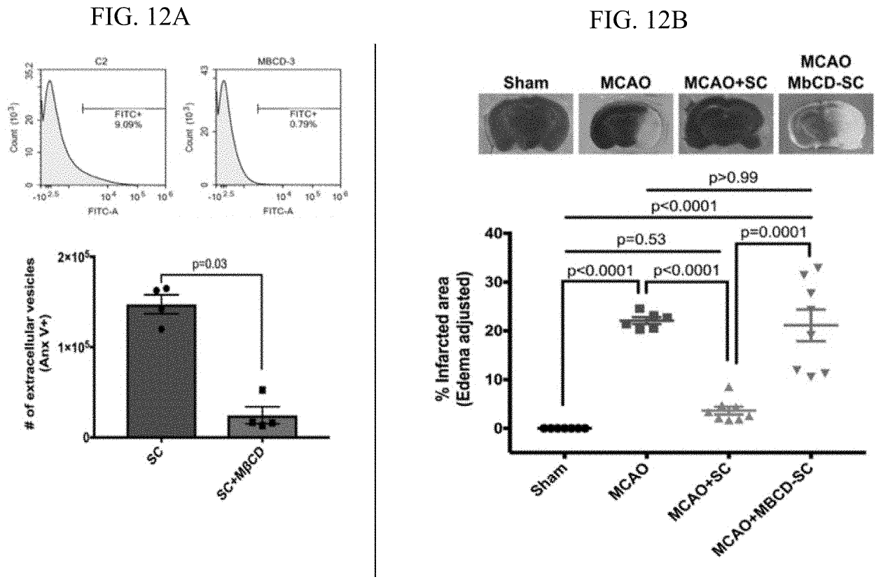

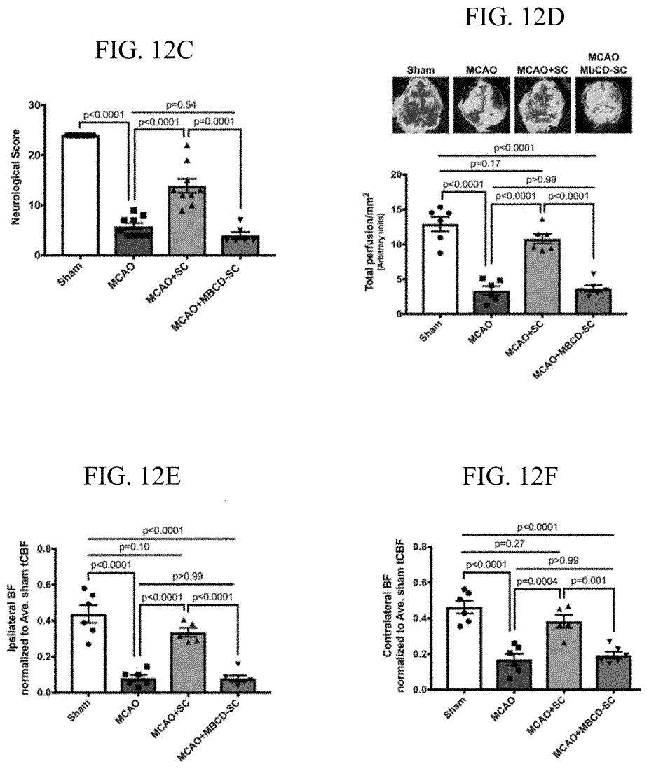

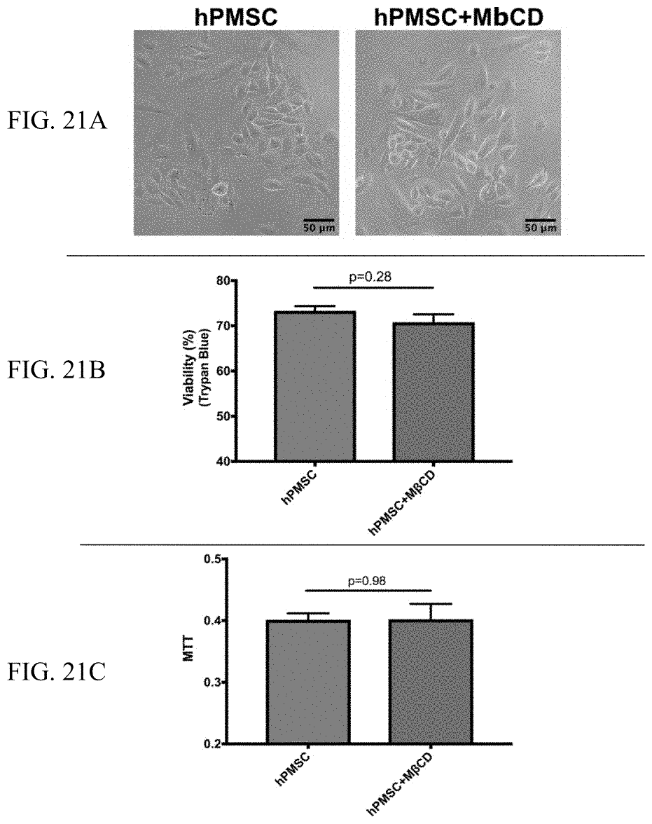

Extracellular vesicles (EVs) are biological vesicles released by cells that contain molecules involved in cell communication, repair and differentiation. Cholesterol is an important structural component of EVs and also regulates EV functional properties. To investigate the possible participation of hPMSC-derived EVs in the enhanced MCAO outcomes seen with hPMSC administration, the inventors blocked the formation/release of EVs from stem cells using 10 mM methyl-beta cyclodextrin (MbCD), which is known to be a non-toxic cholesterol chelating agent. The inventors did not observe morphological changes in MbCD-treated hPMSCs (FIG. 21A); trypan blue staining found no differences in cell viability (p=0.28; FIG. 21B). Further, to detect cell stress in response to exposure to MbCD, the inventors also performed MTT staining on MbCD-treated hPMSCs. The inventors observed that MbCD-treated hPMSC metabolized MTT similar to control hPMSCs (p=0.98; FIG. 21C), consistent with 10 mM MbCD not provoking toxicity within hPMSCs after 2 h incubation time. Lastly, flow cytometric analysis showed a significant decrease (p=0.03, Mann Whitney U test) in the numbers of EVs released from MbCD-treated hPMSCs compared to untreated hPMSCs (FIG. 12A). Unlike hPMSC treatment, the inventors observed that MCAO mice injected with MbCD-treated hPMSCs (21.11±2.63) failed to show protection against MCAO injury (22.08±2.83, p>0.99; FIG. 12B). Similarly, neurological deficits (4±1.77, p=0.54; FIG. 12B) and CBF reductions (3.66±1.05, p>0.99; FIGS. 12D-12F) were similar to untreated MCAO (neurological score: 5.77±1.19 and blood flow: 3.36±1.05). Therefore, hPMSC-derived EVs appear to represent key mediators of SC-based protection after stroke.

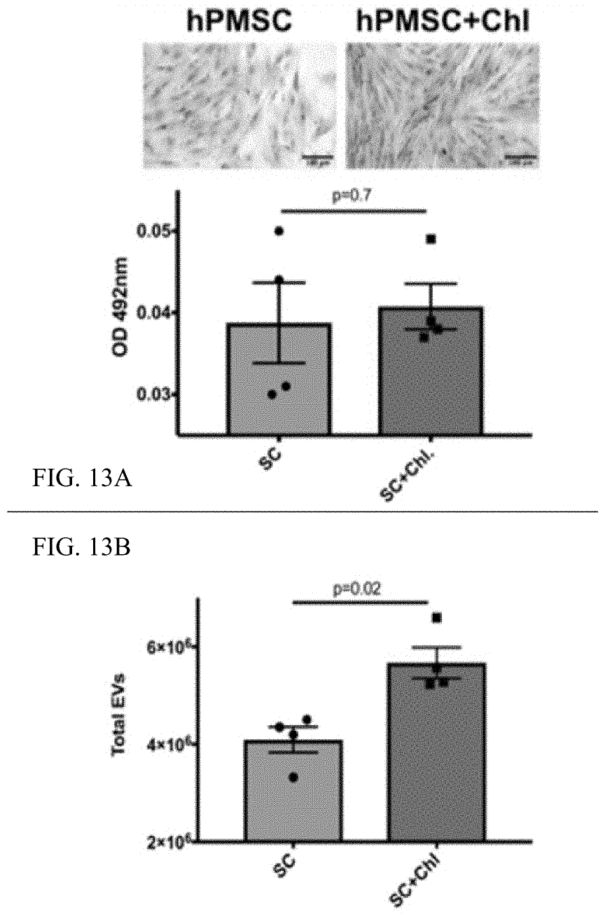

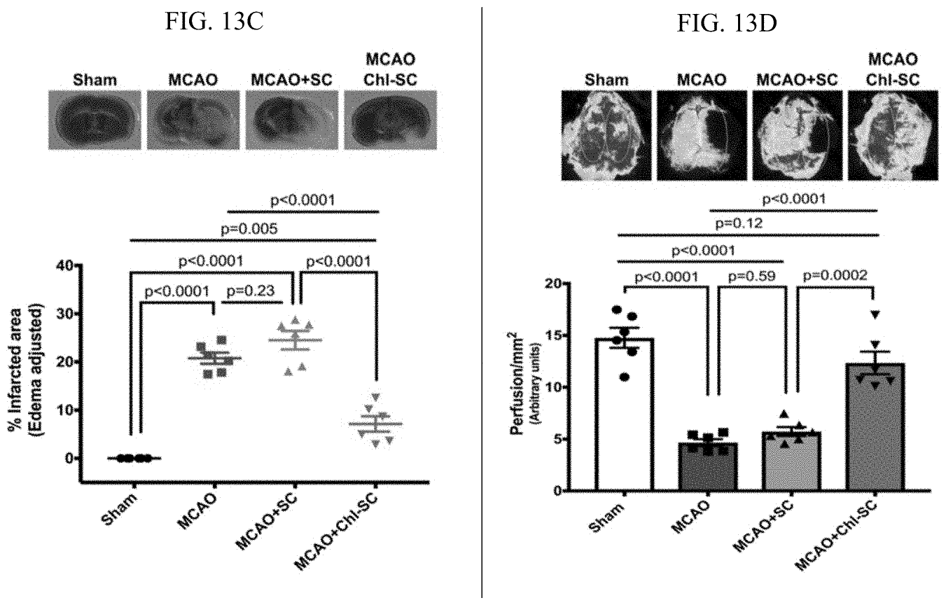

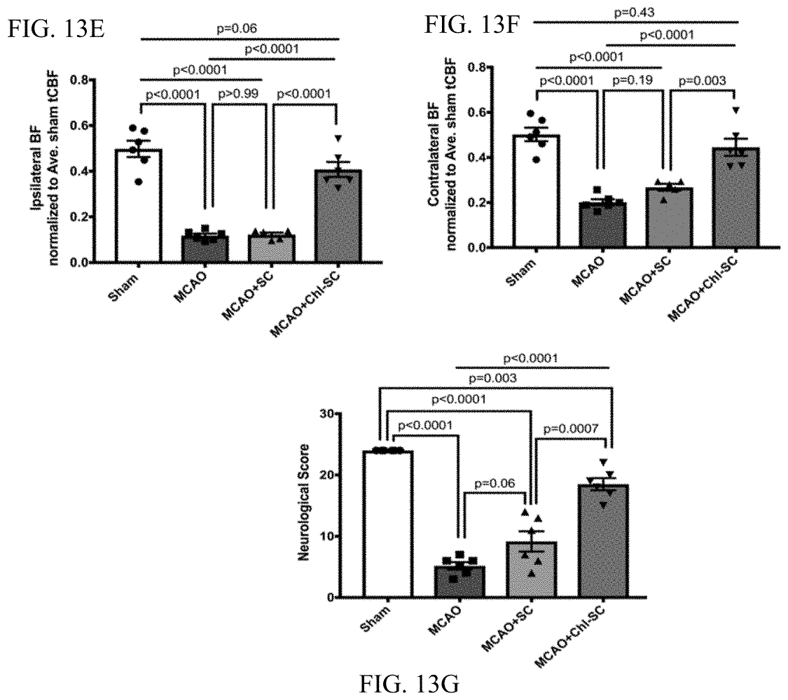

Cholesterol/lipid supplementation augmented protective potential of hPMSCs in the MCAO mice: To test how cholesterol status might contribute to the formation and release of EVs, the inventors treated hPMSC with cholesterol-lipid supplemented media and evaluated the cholesterol content of these cells using Oil Red-O staining. The inventors did not find changes in the lipid and/or intracellular cholesterol content in treated cells (p=0.7; FIG. 13A), however, flow cytometry did reveal significant increases in the numbers of EVs released from hPMSCs by 72 h after cholesterol-lipid treatment (p=0.02; FIG. 13B). This suggested that cholesterol provided to these cells was processed into EVs, which was observed by flow cytometry analysis. To evaluate roles of cholesterol-lipid supplementation in hPMSC-mediated protection, 1×10⁵ cholesterol-supplemented hPMSCs or non-supplemented cells were IP-injected into MCAO treated mice upon reperfusion after 1-hour ischemia. Strikingly, this lower number of cholesterol-supplemented hPMSCs (20% of the number used previously) (FIG. 10) still significantly prevented MCAO brain injury (7.13±2.24, p<0.0001), while the same number of untreated hPMSC (1×10⁵) was insufficient to provide protection (24.52±2.24, p=0.23; FIG. 13B). Behavioral performance (18±1.65, p<0.0001; FIG. 13C) and blood perfusion (12.34±0.99, p<0.0001; FIGS. 13D-13F) were also significantly improved in the low (20%) dosed cholesterol-lipid supplemented hPMSC group. Consequently, cholesterol-lipid supplementation was found to significantly enhance the protective capacity of hPMSC in MCAO, apparently through greater formation/release of EV and/or a more potent version of the EV.

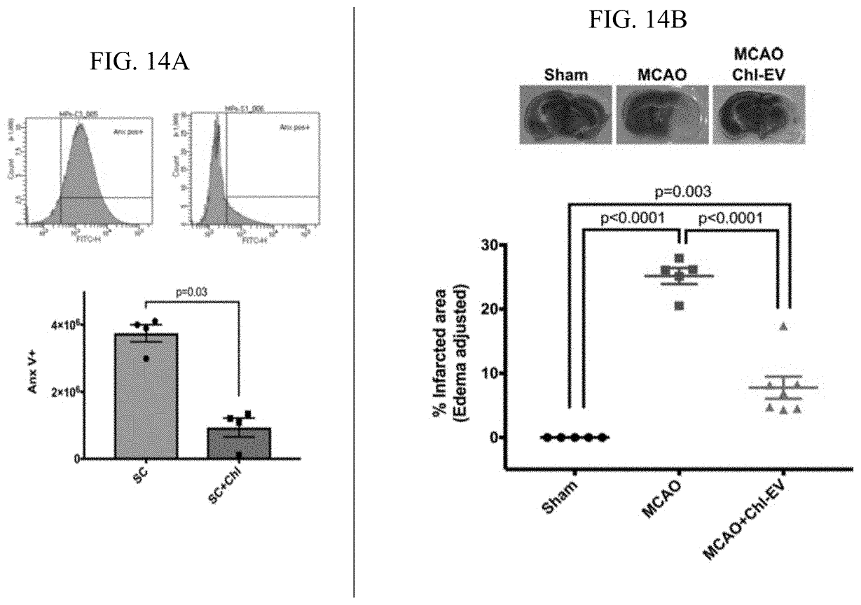

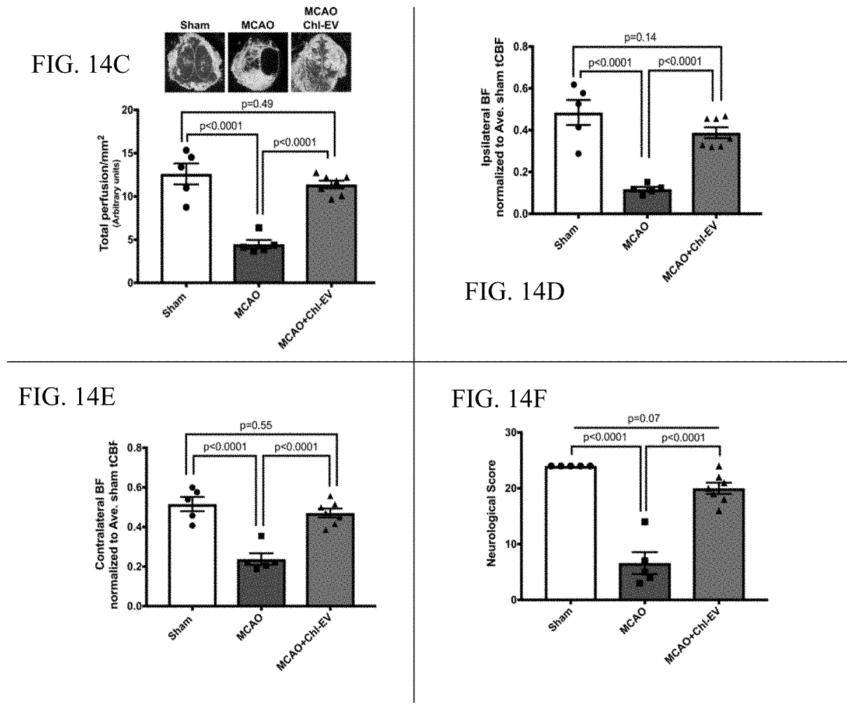

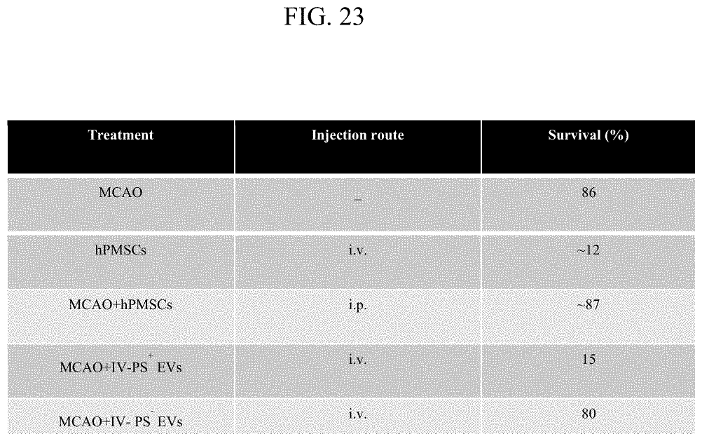

Interestingly, flow cytometric analysis of EVs collected from cholesterol-hPMSCs revealed a significant decrease in annexin-V positive EVs (p=0.03; FIG. 14A), indicating a net reduction in phosphatidylserine (PS) presentation on EVs outer surface. Prior studies confirm PS as a potent pro-coagulant factor, which recruits coagulation activators e.g. tissue factor (TF) that activate factor X to generate thrombin. Such induction of coagulation may explain in part the inventors' low rate of survival (15%) after IV injection of untreated hPMSC-derived EVs into MCAO mice (n=7) (FIG. 23). To study whether cholesterol supplementation of hPMSCs might overcome IV-related complications, PS⁻ EVs, derived from cholesterol-treated hPMSCs (2×10⁶) (i.e. EVs that did not bind Annexin-V) were sorted using FACS and IV-injected into MCAO mice. The inventors next investigated the effect of cholesterol supplementation on the safety and benefits of hPMSCs derived EVs by comparing the MCAO group with MCAO mice IV-injected with EVs isolated from cholesterol-treated hPMSCs (PS⁻ EVs). By comparison 80% of mice (n=10) IV injected with PS⁻ EVs survived (FIG. 23). FIG. 14 shows that tissue injury was significantly reduced from 25.17±1.25 in MCAO to 7.76±1.73 in the cholesterol-treated hPMSC PS⁻ EV group with MCAO (p<0.0001; FIG. 14B); blood perfusion (11.38±0.44, p<0.0001; FIGS. 14C-14E) and behavior (20±1, p<0.0001; FIG. 14F) were both significantly improved.

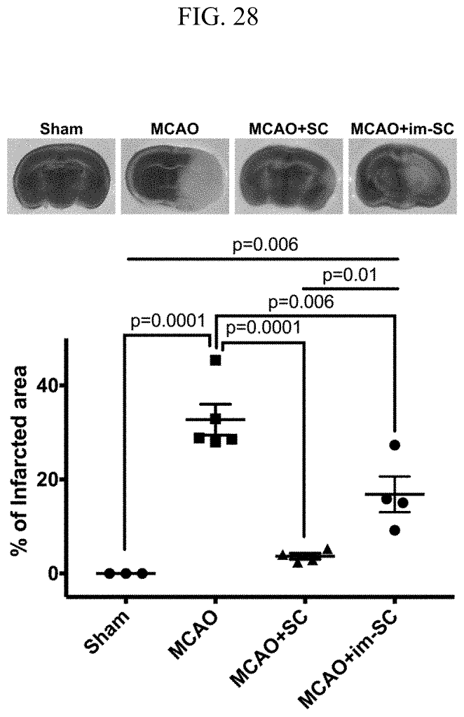

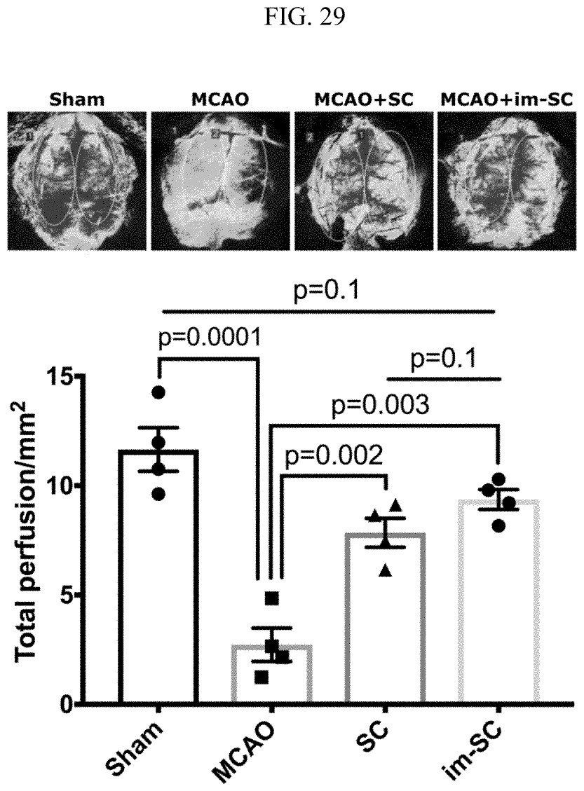

Immortalized human Placenta Mesenchymal Stem Cells (IhPMSC/IPSC) are protective in MCAO model: In FIG. 28, in the MCAO model of stroke injury, immortalized stem cells reduce the fraction of the infarcted region of the ipsilateral hemisphere (MCAO+im-SC, p=0.006) nearly as effectively as non-immortalized stem cells (MCAO+SC, p=0.0001) compared to non-treated stroke model mice (MCAO). In FIG. 29, in the MCAO model of stroke injury, immortalized stem cells maintain the total perfusion of the infarcted region of the ipsilateral hemisphere (MCAO+im-SC, p=0.003) more effectively than non-immortalized stem cells (MCAO+SC, p=0.002) compared to non-treated stroke model mice (MCAO).

DISCUSSION: Here, the inventors report for the first time that hPMSC abundantly express ACE2 protein (FIG. 2A). Compared to endothelial cells, a cell type which specifically expresses ACE2, we found >3-fold greater expression in hPMSCs. Furthermore, mice injected IP with hPMSC, which were pre-treated with the specific ACE2 inhibitor (10 mM) MLN-476035 or lentivirus-shRNA-ACE2, no longer showed protection against MCAO, with tissue injury and neurological behavior similar to that seen in untreated MCAO (FIGS. 1 & 3). The inventors also demonstrated that cerebral blood flow and infarct size in stroke were significantly improved by IP administration of hPMSC in a vascular MasR dependent fashion (FIGS. 7 & 8). These in vivo data provide substantial evidence which supports that hPMSC-based protection in MCAO is mediated through the ACE2/Ang 1-7/MasR axis of RAS. Although the protective effects of ACE2/Ang1-7 have been previously investigated, those studies concluded that the peptides had technical limitations, for example stating intracerebroventricular administration 7 days prior to induction of the ischemic stroke was required for any protection and that immediate protection was not observed—these conclusions are in contradistinction to the inventors' findings. Investigations by others stated that any therapeutic effect required administration before stroke (endothelin-1 model) or that continuous delivery is required—neither of which bears out with the inventors exhaustive and extensive experiments. However, the inventors acknowledge that post-translational mechanisms such as disintegrin and metalloproteinase domain-containing protein 17 (ADAM17)-mediated shedding and AT₁R-dependent internalization have been shown to limit the beneficial effects of ACE2. To reconcile the results from other previous investigators and the inventors' results, the inventors propose the hPMSC EV, which carry the hPMCS derived ACE2, confers a promotion and/or protection to the ACE2 by its envelope and/or other chemicals packaged in the EV, such as, for example, preventing disintegrin and ADAM17 from limiting the effectiveness of ACE2, and with packaged chemicals such as ACE1, or chymase which promotes the beneficial processing of angiotensin 1. Further, the inventors propose that ACE2 derived from hPMSC EVs may itself be more potent and protective. While purifying ACE2 protein from hPMSC EVs for therapeutic administration is contemplated and considered part of the disclosed invention, the inventors expect that ACE2 being delivered as part of the EVs may be superior in terms of biological persistence, especially considering that small proteins are cleared much faster large species.

With respect to COVID-19 biology, it was shown that ACE2 catalytic capacity is lost upon SARS-CoV-2 penetration of endothelial cells leading to derangement of several endothelial regulated functions including vasoregulation, inflammation and thrombosis. These observations are relevant to ACE2 importance in stroke mechanism under normal conditions. In addition, COVID-19 infection of endothelial cells can result in cerebral endothelial dysfunction, inflammation and heightened pro-coagulant state, culminating in the intensified microvascular stroke pathology which is often seen in COVID-19.

The inventors have now demonstrated that hPMSC-derived ACE2 protects against ischemic injury in MCAO stroke. Based on evidence of suppression of endothelial ACE2 by SARS-CoV-2, restoration of ACE2 via hPMSC may provide an innovative and promising approach to maintain ACE2-dependent vascular homeostasis in this disease state. Future studies will elucidate any additional protective mechanisms of hPMSC-derived ACE2 in the setting of brain vascular endothelium in the setting of SARS-CoV-2 infection.

Stroke remains the leading cause of neurologically-based morbidity worldwide (2) with thromboembolic strokes accounting for 87% of total stroke incidence. In ischemic stroke, IRI at the time of, and following therapeutic restoration of CBF often mobilizes intracerebral inflammatory mediators that impair BBB, activate endothelial cells and disturb normal CBF, all of which greatly intensify stroke severity (49). Pharmaceutical interventions for stroke are now limited to two FDA-approved approaches: t-PA and anti-platelet therapies (aspirin/clopidogrel). While these treatments aim to restore blood flow to the brain, their clinical use does not halt the initiation and progression of cerebral reperfusion injury and each carries serious risks for hemorrhage. The lack of highly effective and safe therapies for the acute phase of stroke still demands investigation towards alternative therapeutic approaches for limiting stroke injury using stroke models e.g. MCAO.

The placenta represents an important and highly practical source of MSC for SCT. Placentas contain extremely high numbers of stem cells, with no need for invasive recovery procedures; placentas also lack ethical considerations encountered with fetal tissues.

Here, the inventors' hPMSC were plastic adherent, CD34⁽⁻⁾, CD10⁽⁺⁾, CD200⁽⁺⁾ and CD105⁽⁺⁾ cells (FIGS. 16A-16G). In setting up the inventors' study, the inventors considered potential difficulties associated with xenografting, where host immune response could reject cells from non-human sources (such as murine or porcine cells). The immunomodulatory effects and low immunogenicity of human placental derived stem cells (hPMSC) found these cells to be useful candidates for allogenic transplantation and clinical application in regenerative medicine. Therefore, that these cells are effective in animal models, evidence their benefits in human clinical treatment.

While SCT is commonly used chronically, early and effective limitation of initial stroke injury still represents the greatest opportunity to successfully manage injury, thereby limiting the degree of tissue repair that would otherwise be required later. However, less is known about the benefits of acute (vs chronic) hPMSC administration and the underlying mechanistic basis of stem cells in acute protection. The inventors' present disclosed experiments and result represents the first step in such investigations, by determining whether and to what extent hPMSCs (when administered upon reperfusion) could acutely protect the brain in the murine MCAO model.

Despite its promise, SCT has nonetheless encountered several noteworthy considerations regarding their clinical application. When stem cells are administered intravenously (IV), only a very small fraction (<1%) actually penetrate the brain by 24 h. In this study, the inventors have studied stem cells within the peritoneum, blood and brain (FIGS. 20A-20C). The inventors monitored IP-injected hPMSCs in the circulation and found a maximal number of cells appearing in the vascular compartment at 6 hours following administration; this corresponded to only <1% of injected cells (5000 cells in the 2.5 ml of blood/5×10\hat{ }5 cells given) (FIG. 20B). Although the inventors could not identify a final destination of injected hPMSCs, based on the inventors' knowledge, the spleen and lungs are likely additional end points.

An important limitation of stem cell therapy has been IV administration, which can too often trigger intravascular coagulation and risks of injury or death. In the inventors' hands, only 12% of ‘normal’ mice survived IV injection of hPMSC (n=17) even without receiving MCAO. By comparison, the inventors' pilot studies showed that 87% of MCAO-stressed mice survived after IP injection of hPMSCs (n=15), nearly identical to the survival rate in non-hPMSC treated MCAO mice (86%; 18 mice of 21) (FIG. 23). Therefore, the inventors abandoned IV administration of hPMSCs early on in favor of IP delivery. Remarkably, the inventors found that IP administration of 5×10⁵ hPMSCs at the beginning of reperfusion provided extremely potent and highly significant protection against tissue injury in the MCAO model of stroke (FIG. 10A). To the inventors' knowledge, the inventors' model for the first time shows that unlike IV administration, intraperitoneal-hPMSC administration is highly effective, safe and well-tolerated as a therapeutic approach in MCAO (FIG. 23). Most strikingly, and mechanistically, hPMSC-treated mice subjected to MCAO showed a significant preservation of CBF compared to MCAO-treated mice at 24 h (FIGS. 10B-10D). This pattern also found maintenance of normal blood flow to the ischemic brain hemisphere when hPMSCs were administered following MCAO, a result that was also associated with significantly maintenance of surviving neurons (shown by Nissl staining) (FIGS. 10E-10I) and protection of neurological function (FIG. 10E). The inventors' observation that there was a severe CBF decrease in ipsilateral hemisphere of MCAO mice stands in contrast to some findings in the literature which reflect when blood flow measures were recorded. Several previous stroke studies have measured CBF immediately after occlusion (i.e. at the beginning of reperfusion). However, in the inventors' study, the inventors performed Laser Speckle imaging of blood flow 24 hours after reperfusion. The severe reduction in contralateral CBF of MCAO group in the inventors' study appears to relate to an ongoing and progressive vasoconstrictor provoked by ischemia/reperfusion injury. In support of this concept, the inventors observed that cerebrovascular vasoconstriction and injury had not occurred at 4 hours (FIG. 15A); that is to say, loss of perfusion had not yet taken place by 4 hours. Therefore, maintenance of blood flow using hPMSCs could still be effective at this point and later in the course of therapy. Therefore, hPMSCs are evidenced to provide protection for stroke for at least the 4 hr reperfusion window after the stroke. How much later after reperfusion (i.e. beyond the 4 hr reperfusion ‘window’) hPMSCs may provide protection for stroke, currently the major limitation to administration of t-PA in stroke patients, has yet to be determined.

The inventors have also obtained several lines of evidence indicating that hPMSC protection against MCAO injury may be EV-dependent. First, the inventors observed elimination of protection against MCAO injury when hPMSCs were pre-treated with the cholesterol chelator MbCD (FIGS. 12A-12F). By depleting membrane cholesterol and disrupting lipid rafts, MbCD inhibits formation and release of EVs. Consequently, cholesterol availability may be an important co-factor needed to generate hPMSC-EVs in stroke protection. Consistent with this, the inventors' studies demonstrated for the first time that cholesterol/lipid supplementation of hPMSCs enhanced the protective efficacy of hPMSCs at least 500% in the inventors; MCAO model. The inventors found that 5×10⁵ hPMSCs were needed to protect the brain against MCAO injury (FIGS. 10A-10L), but 20% of this number of hPMSCs (1×10⁵; FIGS. 13A-13G) was not protective. Interestingly, when this low dose of hPMSC (1×10⁵; FIGS. 13A-13G) had been supplemented with cholesterol/lipid and administered IP, it significantly protected against MCAO induced injury (FIGS. 13A-13G), consistent with cholesterol/lipid-treated hPMSCs being more ‘potent’ (based on an efficacious ‘dose’). The inventors do recognize that the exact mechanisms by which cholesterol supplementation of the hPMSCs affords protection in this model remain unclear but consistent with the inventors' data using MbCD-treated hPMSCs, the inventors' findings support a role for EV formation. This topic remains the subject of intensive and ongoing investigations.

While hPMSCs are an abundant, non-immunogenic and ethically ‘neutral’ source of EVs, EVs purified from hPMSCs had not yet been tested clinically for their protective effects in SCT. Some studies have shown that IV injection of hPMSC-derived EVs may be safe and protective in animal models. One complication of IV administration of hPMSCs (or hPMSCs-derived EVs) has been activation of coagulation pathways triggered by PS exposure and TF binding to the surface of stem cells. The inventors also found a high rate of mortality when hPMSC-derived EVs were given intravenously (FIG. 23). Differences between the inventors' findings and those in the literature could reflect different isolation methods and culture media, species or mouse strain receiving the EVs. With respect to EVs in therapy, the inventors found that cholesterol/lipid treatment of hPMSCs significantly reduced PS on the EV surface (FIGS. 14A-14F). Most importantly IV injection of PS negative-sorted EVs (2×10⁶) from cholesterol/lipid-treated hPMSCs was non-traumatic and again significantly protected the brain against MCAO stroke injury (FIGS. 14A-14F). These findings are consistent with PS status playing a critical role in safety and beneficial effects offered by hPMSC-derived EVs. In terms of scale, 5×10⁵ cells administered to a 27-30 g mouse (adjusted for the mass of a 70 kg human) indicates that an equivalent human IP dose would approach 1×10⁹ cells. While feasible, it could be technically challenging to produce such large amounts of cells. However, if cholesterol/lipid supplementation were employed, it is possible that only 20% of this dose (˜2×10⁸) might be needed (FIGS. 13A-13G). Future therapies for stroke might benefit from the use of cholesterol/lipid treated hPMSCs or EVs from these cells as potent therapeutics to stabilize cerebral perfusion, BBB and functional recovery following stroke.

The inventors have demonstrated that beneficial effects of hPMSC appear to be mediated by the release of EVs. The inventors showed that inhibiting EV formation by hPMSCs (using MbCD) eliminated protection against MCAO injury. However, it has been difficult to track the distribution of EVs upon release from hPMSCs. Several studies have introduced methods for labeling and imaging EVs which might provide a possible tool to study EVs trafficking in vitro and in vivo and may help to define EVs biodistribution, and its relationship to their therapeutic activity.

In the inventors' current study, the inventors show that hPMSCs/EVs prevent a severe drop in CBF 24 h after the ischemic insult (FIGS. 10A-10L and 12A-12F). It is unclear as which exact cellular/molecular mechanisms are affected by hPMSC/EVs treatment to produce this benefit. Several studies indicated that MSCs/EVs exert protective effects by stimulating production of vasodilators e.g., nitric oxide (NO) and prostaglandins which increase local CBF. Besides vasodilators, MSCs/EVs may provide vasoprotection by supporting endothelial and smooth muscle functions in autoregulation and vascular homeostasis. The inventors' data are entirely consistent with these models.

The inventors have demonstrated that Immortalized human Placenta Mesenchymal Stem Cells (IhPMSC /IPSC) are protective. As shown in FIGS. 28 and 29, after being immortalized, hPMSC are nearly as protective or more protective as non-immortalized hPMSC. Thus, the inventors' study unlocks a new source of the scarce resource of hPMSC, while retaining or even improving the functionality of the SCs. The invention thus discloses the use of lentiviral vectors or other approaches, e.g. plasmid or other viral/non-viral vector, to introduce SV40 large large-T antigen and/or catalytic subunit of human telomerase (hTERT) to create permanently or transiently immortalized human placenta derived, autologous, or other origin stem cell as a therapeutic approach to maintain blood flow and preserve tissue integrity. These transforming vectors may be under the control of inducible promoters, such as tetracycline, cumate, rapamycin, FKCsA, ABA, tamoxifen, blue light, riboswitch, human elongation factor 1 promoter (EF1A), CMV promoter, human ubiquitin C promoter (UBC), PGK promoter, VP16, and p65, for example, and can provide greater cell expansion prior to use or more continuous propagation (more passages). The inventors' studies demonstrate a method by immortalization for the creation of sufficient cells for injection and/or use in creating cell-derived extracellular vesicles which can be used directly, or frozen for later therapeutic administration either intravenously or intraperitoneally. Additionally, these immortalized cells enable the creation of much greater generation and recovery of stem cell derived extracellular vesicles. Importantly, the use of immortalized cells makes it possible to obtain large enough numbers of cells for clinical ‘doses’, without the limitation of procuring a large number of placentas, to be generated commercially, which could provide protection against stroke injury. This has been successfully tested in the middle cerebral artery occlusion model of stroke (MCAO) in mice with excellent effect. The inventors found that administration of 500,000 stem cells into the peritoneum of mice after MCAO was significantly protective of tissue structure and neurological outcomes in stroke models compared to non-treated controls.

However, scaling this half million SC mouse ‘dose’ to human clinical therapy could require as many as 1-2 billion cells per treatment, which is possible but possibly difficult to obtain based on typical observed cell proliferation rates. However, these IPMSC grow extremely rapidly, provide apparently equivalent if not better protection as non-immortalized hPMSC cells and represent a novel therapeutic source which could be used as cells or as a source of therapeutic extracellular vesicles. The vesicles derived from these stem cells are also highly effective and therapeutic in the inventors stroke model and may have an improved activity towards maintenance/restoration of blood flow and tissue function. By treating the IhPMSC with different culture conditions (e.g. cholesterol/lipid supplement, diminazene, etc), the phenotype and therapeutic benefit provided by these cells will be further augmented, and an even greater number of therapeutically efficacious treatments may be derived from the limited number of human placentas.

One approach for using the IhPMSC would be to treat individuals with IhPMSC or EVs derived therefrom, when the individuals are seen at emergency departments for thrombotic strokes, particularly when tissue plasminogen activator (tPA) is also administered. This treatment, when given after a stroke, would prevent the progressive brain tissue destruction, loss of function and behavioral and motor disturbances which often accompany stroke even when tPA is administered.

This disclosed approach, includes a completely novel type of cell—the IhPMCS—which can be expanded to provide a therapeutic which preserves normal perfusion and tissue function at time points after reperfusion. Because this approach does not require cells to be given into the bloodstream, it is safer, will not influence blood coagulation and can be commercially accessible for large scale production of this therapeutic approach.

The immortalized PMSCs are cost effective, easy to use and will provide an unlimited supply of this type of cells for research purpose and have therapeutic potential. The immortalization of these therapeutic stem cells is specifically inventive for this technology over what has come before in that these cells can be more easily propagated, substantially as effective or even more effective than non-immortalized hPMSC, and the use of the IhPMSC does not require continuous re-isolation. Additionally, IhPMSC grow more prolifically, which provides more cells per unit surface area, making them more suitable for use in bioreactor culture. Furthermore, the IhPMSCs are useful in generating larger numbers of stem cell-derived extracellular vesicles, which the inventors have found to be highly protective in models of stroke. Immortalized stem cells reduced the area of the brain injured by stroke and maintained blood perfusion to substantially the same extent or more as non-immortalized (aging) stem cells.

The IhPMSCs would be used in therapies for stroke, myocardial infarction, tissue survival after surgery, organ transplantation, skin grafting and other forms of injury characterized by poor tissue perfusion after ischemia (interruption of blood flow) as a result of injury or medical procedure.

The inventors further discovered that the EVs from hPMSCs cultured with cholesterol are as effective via IV or IP route as a comparable number of whole cells of non-cholesterol cultured hPMSCs. Therefore, a dosage of the therapeutic that is administered for a human patient, per 70 kg patient mass, may be between preferably 2.0×10⁸ and 1.0×10¹⁰ EVs, more preferably between 5.0×10⁸ and 5.0×10⁹ EVs, even more preferably 1.0×10⁹ and 4.0×10⁹ EVs. The dosage is most preferably about 2.0×10⁹ EVs per 70 kg patient mass. EVs from cholesterol cultured hPMSCs—preferably cholesterol cultured immortalized hPMSCs—may be packaged and stored in units between 1.0×10⁸ and 5.0×10⁹ EVs, more preferably between 2.5×10⁸ and 2.0×10⁹ EVs, and most preferably between 5×10⁸ and 1.0×10⁹ EVs, as such innovative quantities would allow quick access and sufficient administration in effective dosages of EVs when time is of the essence.

The inventors have also disclosed that the molecular mechanism for this protection involves the activity of angiotensin converting enzyme-2 which is abundantly expressed on the surface of these stem cells. This activity potently converts the inflammatory and vasoconstrictive mediator angiotensin-2 into the vasodilatory and anti-inflammatory mediator angiotensin 1-7 which mediates these effects. This is true for both cell-based therapy and extracellular vesicle-based therapies for immortalized stem cells and non-immortalized stem cells.

In summary, the inventors' study demonstrates that hPMSC administration provides powerful and acute protection against stroke injury when given immediately after reperfusion in the MCAO stroke model. This protection appears to be mediated through ACE2/MasR signaling pathway. Mechanistically, the inventors' study shows that ACE2 products (Ang-1-7 and/or Ang1-9) maintain cerebral blood flow in the post-MCAO brain which prevents development of ischemic reperfusion injury. These key findings of this study have important relevance for therapies for acute stroke treatment. These data also evidence that, with the observed suppression of ACE2 in COVID-19 affected individuals, and the accompanying dysregulation of blood flow and coagulation, such individuals would be responsive to this form of therapy. Thus, the rapid delivery of intraperitoneal hPMSCs and/or EVs and/or IV delivery of cholesterol media grown EVs are evidenced to have applicability in both acute ischemic stroke and vascular injury associated with SARS-CoV-2 infection.

In further summary, the inventors' study demonstrates that protective actions of hPMSC administration are mediated by release of extracellular vesicles which favorably impact CBF restoration in the post-MCAO brain, to potentially provide a highly translatable therapy for human stroke.

In still further summary, the inventors' study demonstrates that Immortalized human Placenta Mesenchymal Stem Cells retain protective functionality, and may, in fact, impart a higher level of protection than non-immortalized human Placenta Mesenchymal Stem Cells.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. The present disclosure also contemplates other embodiments “comprising,” “consisting of” and “consisting essentially of,” the embodiments or elements presented herein, whether explicitly set forth or not. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items, while only the terms “consisting of” and “consisting only of” are to be construed in the limitative sense.

Wherefore, I/we claim:

1. A method of treating a stroke in a human patient comprising:
administering a therapeutic to the patient;
wherein the therapeutic comprises one of immortalized human placenta mesenchymal stem cells (hPMSCs) and a product from the immortalized hPMSCs.

2. The method of claim 1 wherein the hPMSCs are sterol medium cultured hPMSCs.

3. The method of claim 2 wherein the hPMSCs were immortalized using one of a catalytic subunit of human telomerase (hTERT) and a SV40 large T antigen.

4. The method of claim 3, wherein a route of administration is intraperitoneal (IP).

5. The method of claim 4 wherein the therapeutic comprises hPMSCs.

6. The method of claim 4 wherein the therapeutic comprises Extracellular Vesicles (EVs) isolated from the hPMSCs.

7. The method of claim 3, wherein a route of administration is intravenous (IV).

8. The method of claim 7 wherein the therapeutic comprises extracellular vesicles (EVs) isolated from the hPMSCs and the sterol is cholesterol.

9. The method of claim 8, wherein the therapeutic contains substantially no live hPMSCs.

10. The method of claim 9, wherein the therapeutic further contains further comprises one of a mas Receptor (masR) agonists and a reagent to biochemically suppress a clearance of Ang1-7.

11. The method of claim 9, wherein a dosage of the therapeutic that is administered for a human patient is one of (a) 2.0×10⁸ and 1.0×10¹⁰ EVs per 70 kg patient mass, (b) 5.0×10⁸ and 5.0×10⁹ EVs per 70 kg patient mass, (c) 1.0×10⁹ and 4.0×10⁹ EVs per 70 kg patient mass, and (d) 2.0×10⁹ EVs per 70 kg patient mass.

12. The method of claim 1, wherein the stroke is one of occlusive, post-occlusive, hemorrhagic, and transient ischemic injury.

13. A therapeutic product comprising:
extracellular vesicles (EVs) isolated from of immortalized human placenta mesenchymal stem cells (hPMSCs).

14. The therapeutic product of claim 13 wherein the hPMSCs were cultured with cholesterol at least 12 hours before the EVs were isolated from the hPMSCs.

15. The therapeutic product of claim 13 further comprises one of a mas Receptor (masR) agonists and a reagent to biochemically suppress a clearance of Ang1-7.

16. The therapeutic product of claim 14, wherein the EVs are packaged in units of between one of
(a) 1.0×10⁸ and 5.0×10⁹ EVs,
(b) 2.5×10⁸ and 2.0×10⁹ EVs, and
(c) 5.0×10⁸ and 1.0×10⁹ EVs.

17. A method of treating COVID-19 disease vascular injury in a human patient comprising:
administering a therapeutic to the patient;
wherein the therapeutic comprises one of human placenta mesenchymal stem cells (hPMSCs) and a product from the hPMSCs.

18. The method of claim 17, wherein the hPMSCs are immortalized.

19. The method of claim 17, wherein a route of administration is intravenous (IV), the therapeutic comprises extracellular vesicles (EVs) isolated from the hPMSCs, and the therapeutic is substantially free from live hPMSCs.

20. The method of claim 19, wherein the therapeutic further comprises one of a mas Receptor (masR) agonists and a reagent to biochemically suppress a clearance of Ang1-7.

21. The method of claim 18, wherein a dosage of the therapeutic that is administered for a human patient is one of (a) 2.0×10⁸ and 1.0×10¹⁰ EVs per 70 kg patient mass, (b) 5.0×10⁸ and 5.0×10⁹ EVs per 70 kg patient mass, (c) 1.0×10⁹ and 4.0×10⁹ EVs per 70 kg patient mass, and (d) 2.0×10⁹ EVs per 70 kg patient mass, and the vascular injury is and intensified microvascular stroke pathology.
